(19) United States
(12) Patent Application Publication
(10) Pub. No.: US 2023/0400472 A1
(43) Pub. Date: Dec. 14, 2023

(54) COMBINATION OF BIOMARKERS OF PRETERM DELIVERY

(71) Applicants: INSTITUT NATIONAL DE LA SANTE ET DE LA RECHERCHE MEDICALE, Paris (FR); CENTRE NATIONAL DE LA RECHERCHE SCIENTIFIQUE, Paris (FR); UNIVERSITÉ PARIS CITÉ, Paris (FR); ASSISTANCE PUBLIQUE-HOPITAUX DE PARIS, Paris (FR)

(72) Inventors: Céline MEHATS, GENTILLY (FR); Louis MARCELLIN, PARIS (FR)

(21) Appl. No.: 18/033,479

(22) PCT Filed: Oct. 21, 2021
(86) PCT No.: PCT/EP2021/079213

§ 371 Date: Apr. 24, 2023

(30) Foreign Application Priority Data
Oct. 26, 2020 (EP) 20203980.6

(51) Int. Cl. G01N 33/68

(52) U.S. Cl. CPC G01N 33/689; G01N 2333/78; G01N 2333/475; G01N 2800/368; G01N 2800/50

(57) ABSTRACT

The present invention relates to the prognosis and diagnosis of preterm delivery. More specifically, the invention relates to new combination of biomarkers for preterm delivery that enable the accurate prognosis and diagnosis of preterm delivery, and in particular enable the accurate prognosis and diagnosis of preterm delivery before 32 weeks of gestation.

### FIELD OF THE INVENTION

The present invention relates to biomarkers, and particularly, although not exclusively, to biomarkers of preterm delivery. The biomarkers are useful, several weeks or months prior to birth, for distinguishing individuals at risk of experiencing birth before 37 weeks of gestation.

### BACKGROUND OF THE INVENTION

Preterm birth or preterm delivery is defined by birth that takes place before the completion of 37 weeks of gestation. It is estimated that over 15 million babies are born preterm annually. Globally, preterm delivery is one of the leading causes of death for children under the age of five with an estimated of one million preterm delivery-related mortalities. Many of the survivors face a lifetime of challenging disabilities which include learning disabilities and visual and hearing problems. Although neonatology advances in the past decades has increased survival rates for preterm delivery, above 20% of preterm neonates will suffer at least one major disability including chronic lung disease, impaired mental development, cerebral palsy, deafness, or blindness.

There is a significant need to identify pregnant women who are at risk of preterm delivery. In the current paradigms, treatment for high-risk pregnancies involves prophylactic treatment or enhanced surveillance or close monitoring of the pregnancy, which reduces preterm delivery rates. However, in most cases, classification of pregnancies as high-risk is attributed to prior medical history or clinical examinations, identifying only a small subsection of the true high-risk pregnancies prone to preterm delivery. Still, the majority of pregnancies that are prone to preterm deliveries are not identified at early stages and hence early medical intervention for such cases is not possible.

There are several tests in the market for risk assessment of preterm delivery for women presenting risk symptoms. One such example is the Fetal Fibronectin (fFN) test which provides a risk assessment for symptomatic women. Fetal fibronectin is present in vaginal fluid if a preterm delivery is likely to occur. Hence, the fFN test is commonly used in pregnant women with symptoms indicating a possibility for preterm delivery, such as contractions, vaginal bleeding, fluid leaking from the vagina, increased vaginal discharge, backache and cramp in lower abdomen. The strength of the fFN test lies in its high negative predictive value for up to 10 days following the test (i.e. a negative result means that there is a low possibility of preterm labour within the next 7 to days following the test). However, when the fFN test is positive, the results are less conclusive.

Another immunoassay test detecting Insulin-like Growth Factor-Binding Protein-1 (IGFBP-1) or Placental Alpha 1-Microglobulin (PAMG-1) is available on the US market. This immunoassay test is particularly useful for rupture of membranes diagnosis. Indeed, PAMG-1 and IGFBP1 are naturally present in high concentrations in amniotic fluid. The presence of high concentrations of PAMG-1 and IGFBP1 in vaginal fluid, will results of a rupture of membranes, and thus an preterm delivery.

Thus, there is an urgent need for effective identification of pregnancies with high-risk of preterm delivery, in order to have better control of the risk of prematurity, in particular a method which allows anticipated/rapid treatment of pregnant women.

The present invention provides biomarkers and methods for prognosing and/or diagnosing risk of preterm delivery to overcome at least in part some of the disadvantages of the prior art. In particular, the present invention seeks to provide a risk assessment for classification of women with high-risk for preterm delivery several weeks or even months before symptoms of preterm delivery appear. The present invention also allow for accurate, rapid and sensitive prognosis and diagnosis of preterm delivery through a measurement of a combination of specific biomarkers taken from sample of an individual at a single point in time.

### SUMMARY OF THE INVENTION

The present invention provides novel combination of biomarkers for preterm delivery, in particular before 32 weeks of gestation and methods for prognosing and/or diagnosing risk or likelihood of preterm delivery in an individual. Accordingly, the present invention provides a combination of biomarkers comprising COL1A1 and CCN5 for use as biomarkers for preterm delivery, wherein the preterm delivery is a preterm delivery before 32 weeks of gestation. The combination of biomarkers may further comprise one or more additional biomarkers for imminent birth selected from the group comprising ALDH1A1, CCL18, CCL2, CCL13, IL1RL1, CD14, CD163, TNFRSF8 or any combination thereof. In a more advantageously embodiment, the combination of biomarkers comprises COL1A1, CCN5, ALDH1A1, CCL18, CCL2, CCL13, IL1RL1, CD14, CD163 and TNFRSF8 for use as biomarkers for preterm delivery and/or imminent birth.

The invention also provides the use of the combination of biomarkers for preterm delivery, wherein the combination of biomarkers comprises COL1A1 and CCN5. One or more additional biomarkers for imminent birth selected from the group comprising ALDH1A1, CCL18, CCL2, CCL13, IL1RL1, CD14, CD163, TNFRSF8 or any combination thereof may be used in the uses of the invention. In a more advantageously embodiment, the combination of biomarkers comprises COL1A1, CCN5, ALDH1A1, CCL18, CCL2, CCL13, IL1RL1, CD14, CD163 and TNFRSF8 may be used in the uses of the invention.

The invention also provides the use of the combination of biomarkers prognosing and/or for diagnosing, whether an individual is at risk of preterm delivery and/or imminent birth, wherein the combination of biomarkers comprises COL1A1 and CCN5.

One or more additional biomarkers for imminent birth selected from the group comprising ALDH1A1, CCL18, CCL2, CCL13, IL1RL1, CD14, CD163, TNFRSF8 or any combination thereof may be used in the uses of the invention. In a more advantageously embodiment, the combination of biomarkers comprises COL1A1, CCN5, ALDH1A1, CCL18, CCL2, CCL13, IL1RL1, CD14, CD163 and TNFRSF8 may be used in the uses of the invention.

The invention also provides a method for prognosing and/or for diagnosing, whether an individual is at risk of preterm delivery and/or imminent birth, the method comprising:
a) determining the presence and/or amount of a combination of biomarkers for preterm delivery in a sample obtained from said individual, and
b) comparing the presence and/or amount of said combination of biomarkers obtained from step a) to the presence and/or amount of the same combination of biomarkers in a control sample, to identify an increased risk for preterm delivery,
wherein said combination of biomarkers comprises COL1A1 and CCN5.

One or more additional biomarkers for imminent birth selected from the group comprising ALDH1A1, CCL18, CCL2, CCL13, IL1RL1, CD14, CD163, TNFRSF8 or any combination thereof may be used in the methods of the invention.

In a more advantageously embodiment, the sample is a sample of vaginal fluid and the biomarkers of the invention are protein or nucleic acid encoding for said protein.

The presence and/or amount of the biomarkers is determined using an antibody and/or an oligonucleotide specific for said biomarkers. In a more advantageously embodiment, the presence and/or amount of the COL1A1 and CCN5 biomarkers is determined using an antibody and/or an oligonucleotide specific for said COL1A1 and CCN5 biomarkers, and the presence and/or amount of the one or more additional biomarkers is determined using an antibody and/or an oligonucleotide specific for said one or more additional biomarkers.

The invention also provides a method for prognosing and/or for diagnosing, whether an individual is at risk of preterm delivery and/or imminent birth, comprises:
a) determining the presence and/or amount of the combination of biomarkers using an antibody and/or an oligonucleotide specific for the combination of biomarkers in a sample obtained from an individual, and
b) comparing the presence and/or amount of said combination of biomarkers obtained from step a) to the presence and/or amount of said combination of biomarkers in a control sample, to identify an increased risk for preterm delivery and/or imminent birth,
wherein the combination of biomarkers is COL1A1, CCN5, ALDH1A1, CCL18, CCL2, CCL13, IL1RL1, CD14, CD163 and TNFRSF8.

Accordingly, to the invention, the individual is pregnant individual.

Accordingly, to the invention, the preterm delivery is a preterm delivery before 32 weeks of gestation.

The invention further provides a device for carrying out the use of the invention or for use in the method of the invention, which comprises:
a) one or more antibody and/or oligonucleotide specific for the combination of COL1A1 and CCN5 biomarkers for preterm delivery, and optionally
b) one or more antibody and/or specific for the combination of one or more additional biomarkers for imminent birth, wherein the one or more additional biomarkers is selected from the group comprising ALDH1A1, CCL18, CCL2, CCL13, IL1RL1, CD14, CD163 and TNFRSF8, and optionally
c) at least one internal standard.

### DESCRIPTION OF FIGURES

FIG. 1A: Box and whisker plot illustrating the distribution of the values of the biomarker COL1A1 according to the case and control groups. The median value is significantly different between the two groups (p=0.0037): Case group: median 9993.8 pg/ml (927-11185 [794-18111]) (25th−75th p [min-max]); Control group: median 1107.9 pg/ml (568-2165 [52-8694]).

FIG. 1B: Diagram illustrating the values of the biomarker COL1A1 at the time of sampling in women with symptomatic preterm labour as a function of the term of spontaneous delivery. The values in grey correspond to the values of the case group, in black, of the control group.

FIG. 1C: ROC curve illustrating the performance of the biomarker COL1A1 to discriminate childbirth before 32 weeks gestation. The value of the biomarker COL1A1 which confers the best sensitivity and specificity is 3657 pg/ml; Sensitivity=66.7%, 95% CI [35.4-87.9]; Specificity 86%, CI95% [78-91]. PPV: 4.762-CI95% [2.436-9.309]. NPV: 0.388-CI95% [0.153-0.98]; Value under the curve: 0.7933, CI95% [0.70-0.86].

FIG. 1D: Adjusted analysis of the biomarker COL1A1 (covariates: maternal age, cervical length, amniotic sac sacculation, CRP, and cervical constancy). For this analysis (logistic regression), subjects with cervical length>25 mm were included (109 in total) and missing values were imputed. Odds ration=18.3, CI95% [1.815-183.653].

FIG. 2A: Box and whisker diagram illustrating the distribution of the values of the biomarker CCN5 according to the case and control groups. The median value is significantly different between the two groups (p=0.0025): Cas group: median: 6240 pg/ml (4714-10064 [1701-14486]) (25th-75th p [min-max]); Control group: median 2514 pg/ml (1891-3608 [5366-126442-8694]).

FIG. 2B: Diagram illustrating the values of the biomarker CCN5 at the time of sampling in women with symptomatic preterm labour as a function of the term of spontaneous delivery. The values in grey correspond to the values of the case group, in black, of the control group.

FIG. 2C: ROC curve illustrating the performance of the biomarker CCN5 to discriminate childbirth before 32 weeks gestation. The value of the biomarker CCN5 which confers the best sensitivity and specificity is 4714 pg/ml; Sensitivity=77.8%; CI95% [45.3-93.6]; Specificity 89%; CI95% [81.4-93.8]; PPV: 7.1-C195% [3.7-13.7]. NPV: 0.25-CI95% [0.07-0.84]; Value under the curve: 0.8056 CI95% [0.72-0.88].

FIG. 2D: Adjusted analysis of the biomarker CCN5 (covariates: maternal age, cervical length, amniotic sac sacculation, CRP and cervical constancy). For this analysis (logistic regression), subjects with cervical length>25 mm were included (109 in total) and missing values were imputed. Odds ration=22.137-CI95% [3.813-128.523].

FIG. 3: Univariate analysis of immunity-related analytes (CCL2, CCL18, CD14 et CD163). Scatterplots showing the distribution of analyte values by “Spontaneous Delivery” and “Induced Delivery” groups and date of vaginal sample (third quarter (3^{rd} Q) and at term (T)). The dotted line indicates the threshold value. The p values of the statistical tests (two-tailed Mann-Whitney) are indicated for the different groups tested.

FIG. 4: Univariate analysis of immunity-related analytes (IL1RL1, ALDH1A1, CCL13, TNFRSF8). Scatterplots showing the distribution of analyte values by “Spontaneous Delivery” and “Induced Delivery” groups and date of vaginal sample (third quarter (3^{rd} Q) and at term (T)). The dotted line indicates the threshold value. The p values of the statistical tests (two-tailed Mann-Whitney) are indicated for the different groups tested.

FIG. 5A: Box and whisker plot illustrating the distribution of the values of the internal standard stratifin (SFN) according to the case and control groups. The median value is not significantly different between the two groups (p=0.752): Case group: median 2099 pg/ml (1766-3314 [795-5016]) (25th-75th p [min-max]); Control group: median 2363 pg/ml (1363-3249 [0-5532]).

FIG. 5B: Diagram illustrating the values of the internal standard at the time of sampling in women with symptomatic preterm labor as a function of the term of spontaneous delivery. The values in grey correspond to the values of the case group, in black, of the control group.

FIG. 5C: ROC curve illustrating the performance of the internal standard stratifin (SFN) to discriminate childbirth before 32 weeks gestation. Value under the curve: 0.5239, CI95% [0.33-0.71].

FIG. 6: Distribution of biomarkers and subjects according to time to delivery. Heatmap illustrating z-scored abundance of biomarkers COL1A1 (A), CCN5 (B), CCL2 (C), IL1RL1 (D), CD14 (E), TNFRSF8 (F), CCL18 (G) and CD163 (H) in subjects with preterm delivery before 32 weeks of gestation.

FIG. 7: ROC curve illustrating the performance of the combination of biomarkers (COL1A1, CCN5, ALDH1A1, CCL18, CCL2, CCL13, IL1RL1, CD14, CD163 and TNFRSF8) to discriminate childbirth before 32 weeks of gestation.

FIG. 8: ROC curve illustrating the performance of the combination of biomarkers (COL1A1, CCN5, CCL2, IL1RL1, CD14, TNFRSF8, CCL18 and CD163) to discriminate childbirth within 7 days after preterm labor diagnosis.

FIG. 9: Distribution of biomarkers and subjects according to time to delivery. Heatmap illustrating z-scored abundance of biomarkers COL1A1 (A), CCN5 (B), CCL2 (C), IL1RL1 (D), CD14 (E), TNFRSF8 (F), CCL18 (G) and CD163 (H) in subjects.

FIG. 10: Plot showing biomarkers abundance in subjects according to gestational age at admission and delay until delivery. Concentrations are higher in CVFs of subjects who delivered within 7 days, regardless of gestational age at admission.

FIG. 11: A) Heatmap illustrating z-scored abundance of biomarkers CCL2 (C), IL1RL1 (D), CD14 (E), TNFRSF8 (F), CCL18 (G) and CD163 (H) and negative controls TGFa (X) and CD66a (Y) in cervical-vaginal fluids of low-risk pregnant women: CVF were sampled during delivery at term. B) Hierarchical clustering of the subjects according to the biomarkers abundance.

### DETAILED DESCRIPTION

The present invention allows for the rapid, sensitive, and accurate diagnosis or prognosis of preterm delivery using one or more samples obtained from an individual at a single time point (“snapshot”) or during the course of disease progression. Preterm birth or preterm delivery may be diagnosed or prognosed prior to the onset of clinical symptoms, and/or as subsequent confirmation after the onset of clinical symptoms. Accordingly, the present invention allows for more effective therapeutic intervention and/or diagnosis in the pre-symptomatic stage.

The present invention relates to a combination of biomarkers comprising COL1A1 and CCN5 for use as biomarkers for preterm delivery, wherein the preterm delivery is a preterm delivery before 32 weeks of gestation.

As used herein, the term “combination” may be used to refer to a mixture of at least two different biomarkers. Advantageously, the combination may comprise at least three different biomarkers, advantageously at least four different biomarkers, advantageously at least five different biomarkers, advantageously at least six different biomarkers, advantageously at least seven different biomarkers, advantageously at least eight different biomarkers, advantageously at least nine different biomarkers, advantageously at least ten different biomarkers.

As used herein, the term “imminent childbirth” or “imminent birth” or “imminent delivery” may be used to refer to a delivery which will occur within less than or equal to 7 days.

As used herein “preterm birth” or “preterm delivery” includes the delivery of a baby prior to full gestation. For example, delivery of the baby less than 37 weeks of gestation is considered a preterm delivery. The term preterm delivery is synonymous with preterm delivery and premature delivery. According to the present invention, a preterm delivery occurs when the delivery of the baby is before 37 weeks of gestation, advantageously before 36 weeks of gestation, advantageously before 35 weeks of gestation, advantageously before 34 weeks of gestation, advantageously before 33 weeks of gestation, advantageously before 32 weeks of gestation, advantageously before 31 weeks of gestation, advantageously before 30 weeks of gestation, advantageously before 29 weeks of gestation, advantageously before 28 weeks of gestation. “Preterm birth” or “preterm delivery” can be used interchangeably. According to the present invention, a preterm delivery occurs when the delivery of the baby is before 32 weeks of gestation.

As used herein, an “individual” is an animal, advantageously a mammal, more advantageously a human. The terms “individual” “subject” and “patient” are used interchangeably herein. Advantageously, the individual is a pregnant woman at risk for preterm delivery, in particular an individual at risk of preterm delivery before 32 weeks of gestation, and benefits from the methods described herein.

As used herein, the term “biomarker” or “biomarkers” may be used to refer to a naturally-occurring biological molecule present in a sample obtained from pregnant individual at varying concentrations and that may be isolated from, or measured in the sample, and which is particularly useful in prognosis and/or diagnosis the risk of preterm delivery, advantageously useful in prognosis and/or diagnosis the risk of preterm delivery before 32 weeks of gestation. For example, the biomarker can be a protein and a fragment thereof, a peptide, a polypeptide, a proteoglycan, a glycoprotein, a lipoprotein, a carbohydrate, a lipid, a nucleic acid, an organic on inorganic chemical, a natural polymer, and a small molecule. Furthermore, a biomarker can be the entire intact molecule, or it can be a portion thereof that may be partially functional or recognized, for example, by an antibody or other specific binding protein. A biomarker is considered to be informative if a measurable aspect or characteristic of the biomarker is associated with a given state of an individual, such as preterm delivery. Such a measurable aspect or characteristic may include, for example, the presence, absence, amount or concentration of the biomarker in the biological sample from the individual and/or its presence as part of a profile of biomarkers. Such a measurable aspect of a biomarker is defined herein as a “feature.” For example, the presence of a biomarker may be a feature. As another example, the amount of a biomarker in a sample, or the amount of a biomarker in a sample compared with a control or reference sample may be a feature. A feature may also be a ratio of two or more measurable aspects of biomarkers, which biomarkers may or may not be of known identity, for example.

The biomarkers disclosed herein were identified in a meta-data analysis, and subsequently demonstrated in patient-derived samples. Biomarkers disclosed herein differentiate samples from term and preterm individuals, weeks or months before the individual is symptomatic. Such biomarkers may be useful for identifying an individual at risk of preterm delivery, and thus may be useful for guiding clinical decisions such as the initiation of treatment to prolong gestation and/or prevent or reduce the risk of preterm delivery. Advantageously, the biomarkers are proteins or nucleic acids issued from motherly tissues and not from the fetal tissue.

In one embodiment, the biomarker can be a protein or a nucleic acid encoding for a protein present in higher or lower amounts in an individual at risk of preterm delivery relative to the amount of the same biomarker in an individual who did not experience preterm delivery. In advantageously embodiment, the biomarker is a protein or a nucleic acid encoding for a protein.

As disclosed herein, COL1A1 and CCN5 are biomarkers of preterm delivery. Surprisingly, the inventors have shown that the increased amount of the specific combination of biomarkers, and in particular the increased amount of the combination of COL1A1 and CCN5 biomarkers indicates that the individual is at risk of preterm delivery. Advantageously, the inventors have shown that the increased amount of the specific combination of biomarkers, and in particular the increased amount of the combination of COL1A1 and CCN5 biomarkers indicates that the individual is at risk of preterm delivery before 32 weeks of gestation. Advantageously, the inventors have shown that when the increased amount of the biomarker COL1A1 is higher than 3657,000 pg/ml in the sample of an individual and the increased amount of the biomarker CCN5 is higher than 4714,000 pg/ml in the sample of an individual, the individual is at risk of preterm delivery. Advantageously, the inventors have shown that when the increased amount of the biomarker COL1A1 is higher than 3657,000 pg/ml in the sample of an individual and the increased amount of the biomarker CCN5 is higher than 4714,000 pg/ml in the sample of an individual, the individual is at risk of preterm delivery before 32 weeks of gestation.

As used herein “COL1A1” may refer to the collagen type I alpha 1 chain gene or to the collagen alpha-1(I) chain protein. According to the present invention, “COL1A1” or “collagen type I alpha 1 chain” or “alpha-1 type I collagen” or “alpha1(I) procollagen” or “collagen alpha 1 chain type I” or “collagen alpha-1(I) chain preproprotein” or “collagen of skin, tendon and bone” or “pro-alpha-1 collagen type 1” or “type I procollagen alpha 1 chain” can be used interchangeably.

As used herein “CCN5” may refer to the cellular communication network factor 5 gene or to the CCN family member 5 protein. According to the present invention, “CCN5” or “WISP2” or “WNT1 inducible signaling pathway protein 2”, or “connective tissue growth factor-like protein” or “connective tissue growth factor-related protein 58” can be used interchangeably.

In particularly advantageous embodiment, the combination of biomarkers of the invention solely comprises COL1A1 and CCN5 as biomarkers for preterm delivery, advantageously as biomarkers for preterm delivery before 32 weeks of gestation. In particularly advantageous embodiment, the combination of biomarkers of the invention contains COL1A1 and CCN5 as biomarkers for preterm delivery, in particular the risk of preterm delivery before 32 weeks of gestation.

In one embodiment, the combination of biomarkers of the invention may further comprises one or more additional biomarkers for imminent birth selected from the group comprising ALDH1A1, CCL18, CCL2, CCL13, IL1RL1, CD14, CD163, TNFRSF8 or any combination thereof.

Advantageously, the inventors have shown that the increased amount of one or more additional biomarkers selected from the group comprising COL1A1, CCN5, ALDH1A1, CCL18, CCL2, CCL13, IL1RL1, CD14, CD163 and TNFRSF8 may indicate that the individual is at risk of imminent birth. Advantageously, the inventors have shown that ALDH1A1, CCL18, CCL2, CCL13, IL1RL1, CD14, CD163 and TNFRSF8 are biomarkers for imminent spontaneous childbirth.

Advantageously, the inventors have shown that when the increased amount of the biomarker ALDH1A1 is higher than 1171,000 pg/ml in the sample of an individual, the individual is at risk of imminent birth.

Advantageously, the inventors have shown that when the increased amount of the biomarker CCL18 is higher than 327,300 pg/ml in the sample of an individual, the individual is at risk of imminent birth. Advantageously, the inventors have shown that when the increased amount of the biomarker CCL2 is higher than 93,410 pg/ml in the sample of an individual, the individual is at risk of imminent birth.

Advantageously, the inventors have shown that when the increased amount of the biomarker CCL13 is higher than 3,615 pg/ml in the sample of an individual, the individual is at risk of imminent birth.

Advantageously, the inventors have shown that when the increased amount of the biomarker IL1RL1 is higher than 9319,000 pg/ml in the sample of an individual, the individual is at risk of imminent birth.

Advantageously, the inventors have shown that when the increased amount of the biomarker CD14 is higher than 16514,000 pg/ml in the sample of an individual, the individual is at risk of imminent birth.

Advantageously, the inventors have shown that when the increased amount of the biomarker CD163 is higher than 12791,000 pg/ml in the sample of an individual, the individual is at risk of imminent birth.

Advantageously, the inventors have shown that when the increased amount of the biomarker TNFRSF8 is higher than 3,355 pg/ml in the sample of an individual, the individual is at risk of imminent birth.

As disclosed herein, COL1A1, CCN5, ALDH1A1, CCL18, CCL2, CCL13, IL1RL1, CD14, CD163 and TNFRSF8 are biomarkers of preterm delivery and/or imminent birth. Advantageously, COL1A1, CCN5, ALDH1A1, CCL18, CCL2, CCL13, IL1RL1, CD14, CD163 and TNFRSF8 are biomarkers of preterm delivery before 32 weeks of gestation. Each of these biomarkers may be used alone, in combination with any of the other biomarkers, and/or in combination with one or more additional biomarker for imminent birth as disclosed herein.

As used herein “ALDH1A1” may refer to the aldehyde dehydrogenase 1 family member A1 gene or to the retinal dehydrogenase 1 protein. According to the present invention, “ALDH1A1” or “ALDH class 1” or “ALHDII” or “RALDH 1” or “acetaldehyde dehydrogenase 1” or “aldehyde dehydrogenase 1” or “soluble aldehyde dehydrogenase” or “liver cytosolic” or “epididymis luminal protein 12” or “epididymis luminal protein 9” or “epididymis secretory sperm binding protein Li 53” or “retinaldehyde dehydrogenase 1” can be used interchangeably. As used herein “CCL18” may refer to the C—C motif chemokine ligand 18 gene or to the C—C motif chemokine 18 protein. According to the present invention, “CCL18” or “PARC” or “CC chemokine PARC” or “CC chemokine ligand 18” or “alternative macrophage activation-associated CC chemokine 1” or “chemokine (C—C motif) ligand 18 (pulmonary and activation-regulated)” or “chemokine (C—C) dendritic” or “dendritic cell chemokine 1” or “macrophage inflammatory protein 4” or “pulmonary and activation-regulated chemokine” or “small inducible cytokine A18” or “small inducible cytokine subfamily A (Cys-Cys) member 18 pulmonary and activation-regulated” or “small inducible cytokine subfamily A (Cys-Cys) member 18 pulmonary and activation-regulated” can be used interchangeably.

As used herein “CCL2” may refer to the C—C motif chemokine ligand 2 gene or to the C—C motif chemokine ligand 2 protein. According to the present invention, “CCL2” or “MCP1” or “chemokine (C—C motif) ligand 2” or “monocyte chemoattractant protein-1” or “monocyte chemotactic and activating factor” or “monocyte chemotactic protein 1” or “monocyte secretory protein JE” or “small inducible cytokine A2 (monocyte chemotactic protein 1, homologous to mouse Sig-je)” or “small inducible cytokine subfamily A (Cys-Cys) member 2” or “small-inducible cytokine A2” can be used interchangeably.

As used herein “CCL13” may refer to the C—C motif chemokine ligand 13 gene or to the C—C motif chemokine ligand 13 protein. According to the present invention, “CCL13” or “CK-beta-10” or “chemokine (C—C motif) ligand 13” or “monocyte chemoattractant protein 4” or “monocyte chemotactic protein 4” or “new CC chemokine 1” or “small inducible cytokine subfamily A (Cys-Cys) member 13” or “small-inducible cytokine A13” can be used interchangeably.

As used herein “IL1RL1” may refer to the interleukin 1 receptor like 1 gene or to the interleukin-1 receptor-like 1 protein. According to the present invention, “IL1RL1” or “IL33R”, or “growth stimulation-expressed” or “homolog of mouse growth stimulation-expressed” or “interleukin 1 receptor-related protein” can be used interchangeably.

As used herein “CD14” may refer to the CD14 molecule gene or to the monocyte differentiation antigen CD14 protein. According to the present invention, “CD14” or “myeloid cell-specific leucine-rich glycoprotein” can be used interchangeably. As used herein “CD163” may refer to the CD163 molecule gene or to the scavenger receptor cysteine-rich type 1 protein M130. According to the present invention, “CD163” or “hemoglobin scavenger receptor” or “macrophage-associated antigen” can be used interchangeably.

As used herein “TNFRSF8” may refer to the TNF receptor superfamily member 8 gene or to the tumor necrosis factor receptor superfamily member 8 protein. According to the present invention, “TNFRSF8” or “CD30” or “CD30L receptor” or “Ki-1 antigen” or “cytokine receptor CD30” or “lymphocyte activation antigen CD30” can be used interchangeably.

In one embodiment, the combination of biomarkers of the invention comprises COL1A1, CCN5 and ALDH1A1 as biomarkers for preterm delivery. In one embodiment, the combination of biomarkers of the invention comprises COL1A1, CCN5 and CCL18 as biomarkers for preterm delivery. In one embodiment, the combination of biomarkers of the invention comprises COL1A1, CCN5 and CCL2 as biomarkers for preterm delivery. In one embodiment, the combination of biomarkers of the invention comprises COL1A1, CCN5 and CCL13 as biomarkers for preterm delivery. In one embodiment, the combination of biomarkers of the invention comprises COL1A1, CCN5 and IL1RL1 as biomarkers for preterm delivery. In one embodiment, the combination of biomarkers of the invention comprises COL1A1, CCN5 and CD14 as biomarkers for preterm delivery. In one embodiment, the combination of biomarkers of the invention comprises COL1A1, CCN5 and CD163 as biomarkers for preterm delivery. In one embodiment, the combination of biomarkers of the invention comprises COL1A1, CCN5 and TNFRSF8 as biomarkers for preterm delivery.

In one embodiment, the combination of biomarkers of the invention comprises COL1A1, CCN5, ALDH1A1 and CCL18 as biomarkers for preterm delivery. In one embodiment, the combination of biomarkers of the invention comprises COL1A1, CCN5, ALDH1A1 and CCL2 as biomarkers for preterm delivery. In one embodiment, the combination of biomarkers of the invention comprises COL1A1, CCN5, ALDH1A1 and CCL13 as biomarkers for preterm delivery. In one embodiment, the combination of biomarkers of the invention comprises COL1A1, CCN5, ALDH1A1 and IL1RL1 as biomarkers for preterm delivery. In one embodiment, the combination of biomarkers of the invention comprises COL1A1, CCN5, ALDH1A1 and CD14 as biomarkers for preterm delivery. In one embodiment, the combination of biomarkers of the invention comprises COL1A1, CCN5, ALDH1A1 and CD163 as biomarkers for preterm delivery. In one embodiment, the combination of biomarkers of the invention comprises COL1A1, CCN5, ALDH1A1 and TNFRSF8 as biomarkers for preterm delivery.

In one embodiment, the combination of biomarkers of the invention comprises COL1A1, CCN5, ALDH1A1, CCL18 and CCL2 as biomarkers for preterm delivery. In one embodiment, the combination of biomarkers of the invention comprises COL1A1, CCN5, ALDH1A1, CCL18 and CCL13 as biomarkers for preterm delivery. In one embodiment, the combination of biomarkers of the invention comprises COL1A1, CCN5, ALDH1A1, CCL18 and IL1RL1 as biomarkers for preterm delivery. In one embodiment, the combination of biomarkers of the invention comprises COL1A1, CCN5, ALDH1A1, CCL18 and CD14 as biomarkers for preterm delivery. In one embodiment, the combination of biomarkers of the invention comprises COL1A1, CCN5, ALDH1A1, CCL18 and CD163 as biomarkers for preterm delivery. In one embodiment, the combination of biomarkers of the invention comprises COL1A1, CCN5, ALDH1A1, CCL18 and TNFRSF8 as biomarkers for preterm delivery.

In one embodiment, the combination of biomarkers of the invention comprises COL1A1, CCN5, ALDH1A1, CCL18, CCL2 and CCL13 as biomarkers for preterm delivery. In one embodiment, the combination of biomarkers of the invention comprises COL1A1, CCN5, ALDH1A1, CCL18, CCL2 and IL1RL1 as biomarkers for preterm delivery. In one embodiment, the combination of biomarkers of the invention comprises COL1A1, CCN5, ALDH1A1, CCL18, CCL2 and CD14 as biomarkers for preterm delivery. In one embodiment, the combination of biomarkers of the invention comprises COL1A1, CCN5, ALDH1A1, CCL18, CCL2 and CD163 as biomarkers for preterm delivery. In one embodiment, the combination of biomarkers of the invention comprises COL1A1, CCN5, ALDH1A1, CCL18, CCL2 and TNFRSF8 as biomarkers for preterm delivery.

In one embodiment, the combination of biomarkers of the invention comprises COL1A1, CCN5, ALDH1A1, CCL18, CCL2, CCL13 and IL1RL1 as biomarkers for preterm delivery. In one embodiment, the combination of biomarkers of the invention comprises COL1A1, CCN5, ALDH1A1, CCL18, CCL2, CCL13 and CD14 as biomarkers for preterm delivery. In one embodiment, the combination of biomarkers of the invention comprises COL1A1, CCN5, ALDH1A1, CCL18, CCL2, CCL13 and CD163 as biomarkers for preterm delivery. In one embodiment, the combination of biomarkers of the invention comprises COL1A1, CCN5, ALDH1A1, CCL18, CCL2, CCL13 and TNFRSF8 as biomarkers for preterm delivery.

In one embodiment, the combination of biomarkers of the invention comprises COL1A1, CCN5, ALDH1A1, CCL18, CCL2, CCL13, IL1RL1 and CD14 as biomarkers for preterm delivery. In one embodiment, the combination of biomarkers of the invention comprises COL1A1, CCN5, ALDH1A1, CCL18, CCL2, CCL13, IL1RL1 and CD163 as biomarkers for preterm delivery. In one embodiment, the combination of biomarkers of the invention comprises COL1A1, CCN5, ALDH1A1, CCL18, CCL2, CCL13, IL1RL1 and TNFRSF8 as biomarkers for preterm delivery.

In one embodiment, the combination of biomarkers of the invention comprises COL1A1, CCN5, ALDH1A1, CCL18, CCL2, CCL13, IL1RL1, CD14 and CD163 as biomarkers for preterm delivery. In one embodiment, the combination of biomarkers of the invention comprises COL1A1, CCN5, ALDH1A1, CCL18, CCL2, CCL13, IL1RL1, CD14 and TNFRSF8 as biomarkers for preterm delivery. In one embodiment, the combination of biomarkers of the invention comprises COL1A1, CCN5, ALDH1A1, CCL18, CCL2, CCL13, IL1RL1, CD14, CD163 and TNFRSF8 as biomarkers for preterm delivery.

In one embodiment, the combination of biomarkers of the invention comprises COL1A1, CCN5, CCL18 and CCL2 as biomarkers for preterm delivery. In one embodiment, the combination of biomarkers of the invention comprises COL1A1, CCN5, CCL18 and CCL13 as biomarkers for preterm delivery. In one embodiment, the combination of biomarkers of the invention comprises COL1A1, CCN5, CCL18 and IL1RL1 as biomarkers for preterm delivery. In one embodiment, the combination of biomarkers of the invention comprises COL1A1, CCN5, CCL18 and CD14 as biomarkers for preterm delivery. In one embodiment, the combination of biomarkers of the invention comprises COL1A1, CCN5, CCL18 and CD163 as biomarkers for preterm delivery. In one embodiment, the combination of biomarkers of the invention comprises COL1A1, CCN5, CCL18 and TNFRSF8 as biomarkers for preterm delivery.

In one embodiment, the combination of biomarkers of the invention comprises COL1A1, CCN5, CCL18, CCL2 and CCL13 as biomarkers for preterm delivery. In one embodiment, the combination of biomarkers of the invention comprises COL1A1, CCN5, CCL18, CCL2 and IL1RL1 as biomarkers for preterm delivery. In one embodiment, the combination of biomarkers of the invention comprises COL1A1, CCN5, CCL18, CCL2 and CD14 as biomarkers for preterm delivery. In one embodiment, the combination of biomarkers of the invention comprises COL1A1, CCN5, CCL18, CCL2 and CD163 as biomarkers for preterm delivery. In one embodiment, the combination of biomarkers of the invention comprises COL1A1, CCN5, CCL18, CCL2 and TNFRSF8 as biomarkers for preterm delivery.

In one embodiment, the combination of biomarkers of the invention comprises COL1A1, CCN5, CCL18, CCL2, CCL13 and IL1RL1 as biomarkers for preterm delivery. In one embodiment, the combination of biomarkers of the invention comprises COL1A1, CCN5, CCL18, CCL2, CCL13 and CD14 as biomarkers for preterm delivery. In one embodiment, the combination of biomarkers of the invention comprises COL1A1, CCN5, CCL18, CCL2, CCL13 and CD163 as biomarkers for preterm delivery. In one embodiment, the combination of biomarkers of the invention comprises COL1A1, CCN5, CCL18, CCL2, CCL13 and TNFRSF8 as biomarkers for preterm delivery.

In one embodiment, the combination of biomarkers of the invention comprises COL1A1, CCN5, CCL18, CCL2, CCL13, IL1RL1 and CD14 as biomarkers for preterm delivery. In one embodiment, the combination of biomarkers of the invention comprises COL1A1, CCN5, CCL18, CCL2, CCL13, IL1RL1 and CD163 as biomarkers for preterm delivery. In one embodiment, the combination of biomarkers of the invention comprises COL1A1, CCN5, CCL18, CCL2, CCL13, IL1RL1 and TNFRSF8 as biomarkers for preterm delivery.

In one embodiment, the combination of biomarkers of the invention comprises COL1A1, CCN5, CCL18, CCL2, CCL13, IL1RL1, CD14 and CD163 as biomarkers for preterm delivery. In one embodiment, the combination of biomarkers of the invention comprises COL1A1, CCN5, CCL18, CCL2, CCL13, IL1RL, CD14 and TNFRSF8 as biomarkers for preterm delivery. In one embodiment, the combination of biomarkers of the invention comprises COL1A1, CCN5, CCL18, CCL2, CCL13, IL1RL, CD14, CD163 and TNFRSF8 as biomarkers for preterm delivery.

In one embodiment, the combination of biomarkers of the invention comprises COL1A1, CCN5, CCL2 and CCL13 as biomarkers for preterm delivery. In one embodiment, the combination of biomarkers of the invention comprises COL1A1, CCN5, CCL2 and IL1RL1 as biomarkers for preterm delivery. In one embodiment, the combination of biomarkers of the invention comprises COL1A1, CCN5, CCL2 and CD14 as biomarkers for preterm delivery. In one embodiment, the combination of biomarkers of the invention comprises COL1A1, CCN5, CCL2 and CD163 as biomarkers for preterm delivery. In one embodiment, the combination of biomarkers of the invention comprises COL1A1, CCN5, CCL2 and TNFRSF8 as biomarkers for preterm delivery.

In one embodiment, the combination of biomarkers of the invention comprises COL1A1, CCN5, CCL2, CCL13 and IL1RL1 as biomarkers for preterm delivery. In one embodiment, the combination of biomarkers of the invention comprises COL1A1, CCN5, CCL2, CCL13 and CD14 as biomarkers for preterm delivery. In one embodiment, the combination of biomarkers of the invention comprises COL1A1, CCN5, CCL2, CCL13 and CD163 as biomarkers for preterm delivery. In one embodiment, the combination of biomarkers of the invention comprises COL1A1, CCN5, CCL2, CCL13 and TNFRSF8 as biomarkers for preterm delivery.

In one embodiment, the combination of biomarkers of the invention comprises COL1A1, CCN5, CCL2, CCL13, IL1RL1 and CD14 as biomarkers for preterm delivery. In one embodiment, the combination of biomarkers of the invention comprises COL1A1, CCN5, CCL2, CCL13, IL1RL1 and CD163 as biomarkers for preterm delivery. In one embodiment, the combination of biomarkers of the invention comprises COL1A1, CCN5, CCL2, CCL13, IL1RL1 and TNFRSF8 as biomarkers for preterm delivery.

In one embodiment, the combination of biomarkers of the invention comprises COL1A1, CCN5, CCL2, CCL13, IL1RL1, CD14 and CD163 as biomarkers for preterm delivery. In one embodiment, the combination of biomarkers of the invention comprises COL1A1, CCN5, CCL2, CCL13, IL1RL, CD14 and TNFRSF8 as biomarkers for preterm delivery. In one embodiment, the combination of biomarkers of the invention comprises COL1A1, CCN5, CCL2, CCL13, IL1RL, CD14, CD163 and TNFRSF8 as biomarkers for preterm delivery.

In one embodiment, the combination of biomarkers of the invention comprises COL1A1, CCN5, CCL13 and IL1RL1 as biomarkers for preterm delivery. In one embodiment, the combination of biomarkers of the invention comprises COL1A1, CCN5, CCL13 and CD14 as biomarkers for preterm delivery. In one embodiment, the combination of biomarkers of the invention comprises COL1A1, CCN5, CCL13 and CD163 as biomarkers for preterm delivery. In one embodiment, the combination of biomarkers of the invention comprises COL1A1, CCN5, CCL13 and TNFRSF8 as biomarkers for preterm delivery.

In one embodiment, the combination of biomarkers of the invention comprises COL1A1, CCN5, CCL13, IL1RL1 and CD14 as biomarkers for preterm delivery. In one embodiment, the combination of biomarkers of the invention comprises COL1A1, CCN5, CCL13, IL1RL1 and CD163 as biomarkers for preterm delivery. In one embodiment, the combination of biomarkers of the invention comprises COL1A1, CCN5, CCL13, IL1RL1 and TNFRSF8 as biomarkers for preterm delivery.

In one embodiment, the combination of biomarkers of the invention comprises COL1A1, CCN5, CCL13, IL1RL1, CD14 and CD163 as biomarkers for preterm delivery. In one embodiment, the combination of biomarkers of the invention comprises COL1A1, CCN5, CCL13, IL1RL, CD14 and TNFRSF8 as biomarkers for preterm delivery. In one embodiment, the combination of biomarkers of the invention comprises COL1A1, CCN5, CCL13, IL1RL, CD14, CD163 and TNFRSF8 as biomarkers for preterm delivery.

In one embodiment, the combination of biomarkers of the invention comprises COL1A1, CCN5, IL1RL1 and CD14 as biomarkers for preterm delivery. In one embodiment, the combination of biomarkers of the invention comprises COL1A1, CCN5, IL1RL1 and CD163 as biomarkers for preterm delivery. In one embodiment, the combination of biomarkers of the invention comprises COL1A1, CCN5, IL1RL1 and TNFRSF8 as biomarkers for preterm delivery.

In one embodiment, the combination of biomarkers of the invention comprises COL1A1, CCN5, IL1RL1, CD14 and CD163 as biomarkers for preterm delivery. In one embodiment, the combination of biomarkers of the invention comprises COL1A1, CCN5, IL1RL, CD14 and TNFRSF8 as biomarkers for preterm delivery. In one embodiment, the combination of biomarkers of the invention comprises COL1A1, CCN5, IL1RL, CD14, CD163 and TNFRSF8 as biomarkers for preterm delivery.

In one embodiment, the combination of biomarkers of the invention comprises COL1A1, CCN5, CD14 and CD163 as biomarkers for preterm delivery. In one embodiment, the combination of biomarkers of the invention comprises COL1A1, CCN5, CD14 and TNFRSF8 as biomarkers for preterm delivery. In one embodiment, the combination of biomarkers of the invention comprises COL1A1, CCN5, CD14, CD163 and TNFRSF8 as biomarkers for preterm delivery.

In one embodiment, the combination of biomarkers of the invention comprises COL1A1, CCN5, CD163 and TNFRSF8 as biomarkers for preterm delivery.

In particularly advantageous embodiment, the combination of biomarkers of the invention comprises COL1A1, CCN5, ALDH1A1, CCL18, CCL2, CCL13, IL1RL1, CD14, CD163 and TNFRSF8. Advantageously, the increased amount of a combination of biomarkers comprising COL1A1, CCN5, ALDH1A1, CCL18, CCL2, CCL13, IL1RL1, CD14, CD163 and TNFRSF8 may indicate that the individual is at risk of preterm delivery.

In particularly advantageous embodiment, the combination of biomarkers of the invention solely comprises COL1A1, CCN5, ALDH1A1, CCL18, CCL2, CCL13, IL1RL1, CD14, CD163 and TNFRSF8 as biomarkers for preterm delivery. In particularly advantageous embodiment, the combination of biomarkers of the invention contains COL1A1, CCN5, ALDH1A1, CCL18, CCL2, CCL13, IL1RL1, CD14, CD163 and TNFRSF8 as biomarkers for preterm delivery.

According to the present invention, all the above disclosed combinations of biomarkers are useful as biomarkers for preterm delivery before 32 weeks of gestation.

In one embodiment, the combination of biomarkers of the invention comprises COL1A1, CCN5 and CCL18 as biomarkers for imminent birth. In one embodiment, the combination of biomarkers of the invention comprises COL1A1, CCN5 and CCL2 as biomarkers for imminent birth. In one embodiment, the combination of biomarkers of the invention comprises COL1A1, CCN5 and IL1RL1 as biomarkers for imminent birth y. In one embodiment, the combination of biomarkers of the invention comprises COL1A1, CCN5 and CD14 as biomarkers for imminent birth. In one embodiment, the combination of biomarkers of the invention comprises COL1A1, CCN5 and CD163 as biomarkers for imminent birth. In one embodiment, the combination of biomarkers of the invention comprises COL1A1, CCN5 and TNFRSF8 as biomarkers for imminent birth.

In one embodiment, the combination of biomarkers of the invention comprises COL1A1, CCN5, CCL18 and CCL2 as biomarkers for imminent birth. In one embodiment, the combination of biomarkers of the invention comprises COL1A1, CCN5, CCL18 and IL1RL1 as biomarkers for imminent birth. In one embodiment, the combination of biomarkers of the invention comprises COL1A1, CCN5, CCL18 and CD14 as biomarkers for imminent birth. In one embodiment, the combination of biomarkers of the invention comprises COL1A1, CCN5, CCL18 and CD163 as biomarkers for imminent birth. In one embodiment, the combination of biomarkers of the invention comprises COL1A1, CCN5, CCL18 and TNFRSF8 as biomarkers for imminent birth.

In one embodiment, the combination of biomarkers of the invention comprises COL1A1, CCN5, CCL18, CCL2 and IL1RL1 as biomarkers for imminent birth In one embodiment, the combination of biomarkers of the invention comprises COL1A1, CCN5, CCL18, CCL2 and CD14 as biomarkers for imminent birth. In one embodiment, the combination of biomarkers of the invention comprises COL1A1, CCN5, CCL18, CCL2 and CD163 as biomarkers for imminent birth. In one embodiment, the combination of biomarkers of the invention comprises COL1A1, CCN5, CCL18, CCL2 and TNFRSF8 as biomarkers for imminent birth.

In one embodiment, the combination of biomarkers of the invention comprises COL1A1, CCN5, CCL18, CCL2, IL1RL1 and CD14 as biomarkers for imminent birth. In one embodiment, the combination of biomarkers of the invention comprises COL1A1, CCN5, CCL18, CCL2, IL1RL1 and CD163 as biomarkers for imminent birth. In one embodiment, the combination of biomarkers of the invention comprises COL1A1, CCN5, CCL18, CCL2, IL1RL1 and TNFRSF8 as biomarkers for imminent birth.

In one embodiment, the combination of biomarkers of the invention comprises COL1A1, CCN5, CCL18, CCL2, IL1RL1, CD14 and CD163 as biomarkers for imminent birth. In one embodiment, the combination of biomarkers of the invention comprises COL1A1, CCN5, CCL18, CCL2, IL1RL1, CD14 and TNFRSF8 as biomarkers for imminent birth. In one embodiment, the combination of biomarkers of the invention comprises COL1A1, CCN5, CCL18, CCL2, IL1RL1, CD14, CD163 and TNFRSF8 as biomarkers for imminent birth.

In one embodiment, the combination of biomarkers of the invention comprises COL1A1, CCN5, CCL2 and IL1RL1 as biomarkers for imminent birth. In one embodiment, the combination of biomarkers of the invention comprises COL1A1, CCN5, CCL2 and CD14 as biomarkers for imminent birth. In one embodiment, the combination of biomarkers of the invention comprises COL1A1, CCN5, CCL2 and CD163 as biomarkers for imminent birth. In one embodiment, the combination of biomarkers of the invention comprises COL1A1, CCN5, CCL2 and TNFRSF8 as biomarkers for imminent birth.

In one embodiment, the combination of biomarkers of the invention comprises COL1A1, CCN5, CCL2, IL1RL1 and CD14 as biomarkers for imminent birth. In one embodiment, the combination of biomarkers of the invention comprises COL1A1, CCN5, CCL2, IL1RL1 and CD163 as biomarkers for imminent birth. In one embodiment, the combination of biomarkers of the invention comprises COL1A1, CCN5, CCL2, IL1RL1 and TNFRSF8 as biomarkers for imminent birth.

In one embodiment, the combination of biomarkers of the invention comprises COL1A1, CCN5, CCL2, IL1RL1, CD14 and CD163 as biomarkers for imminent birth. In one embodiment, the combination of biomarkers of the invention comprises COL1A1, CCN5, CCL2, IL1RL, CD14 and TNFRSF8 as biomarkers for imminent birth. In one embodiment, the combination of biomarkers of the invention comprises COL1A1, CCN5, CCL2, IL1RL, CD14, CD163 and TNFRSF8 as biomarkers for imminent birth.

In one embodiment, the combination of biomarkers of the invention comprises COL1A1, CCN5, IL1RL1 and CD14 as biomarkers for imminent birth. In one embodiment, the combination of biomarkers of the invention comprises COL1A1, CCN5, IL1RL1 and CD163 as biomarkers for imminent birth. In one embodiment, the combination of biomarkers of the invention comprises COL1A1, CCN5, IL1RL1 and TNFRSF8 as biomarkers for imminent birth.

In one embodiment, the combination of biomarkers of the invention comprises COL1A1, CCN5, IL1RL1, CD14 and CD163 as biomarkers for imminent birth. In one embodiment, the combination of biomarkers of the invention comprises COL1A1, CCN5, IL1RL, CD14 and TNFRSF8 as biomarkers for imminent birth. In one embodiment, the combination of biomarkers of the invention comprises COL1A1, CCN5, IL1RL, CD14, CD163 and TNFRSF8 as biomarkers for imminent birth.

In one embodiment, the combination of biomarkers of the invention comprises COL1A1, CCN5, CD14 and CD163 as biomarkers for imminent birth. In one embodiment, the combination of biomarkers of the invention comprises COL1A1, CCN5, CD14 and TNFRSF8 as biomarkers for imminent birth. In one embodiment, the combination of biomarkers of the invention comprises COL1A1, CCN5, CD14, CD163 and TNFRSF8 as biomarkers for imminent birth.

In one embodiment, the combination of biomarkers of the invention comprises COL1A1, CCN5, CD163 and TNFRSF8 as biomarkers for imminent birth.

In particularly advantageous embodiment, the combination of biomarkers of the invention comprises COL1A1, CCN5, CCL18, CCL2, IL1RL1, CD14, CD163 and TNFRSF8. Advantageously, the increased amount of a combination of biomarkers comprising COL1A1, CCN5, CCL18, CCL2, IL1RL1, CD14, CD163 and TNFRSF8 may indicate that the individual is at risk of imminent birth.

In particularly advantageous embodiment, the combination of biomarkers of the invention solely comprises COL1A1, CCN5, CCL18, CCL2, IL1RL1, CD14, CD163 and TNFRSF8 as biomarkers for imminent birth. In particularly advantageous embodiment, the combination of biomarkers of the invention contains COL1A1, CCN5, CCL18, CCL2, IL1RL1, CD14, CD163 and TNFRSF8 as biomarkers for imminent birth.

According to the present invention, the biomarker of the invention may be a protein selected from the group comprising COL1A1, CCN5, ALDH1A1, CCL18, CCL2, CCL13, IL1RL1, CD14, CD163, TNFRSF8 or a nucleic acid encoding for one of said proteins.

The present invention also provides the use of a combination of biomarkers for preterm delivery, wherein the combination of biomarkers comprises COL1A1 and CCN5. Advantageously, the combination of biomarkers for its use in preterm delivery comprises COL1A1 and CCN5. Advantageously, the combination of biomarkers for its use in preterm delivery before 32 weeks of gestation comprises COL1A1 and CCN5.

This combination of biomarkers may be used alone and/or in combination with one or more additional biomarker for preterm delivery, advantageously for preterm delivery before 32 weeks of gestation as disclosed herein.

Advantageously, the combination of biomarkers for preterm delivery may further comprise one or more additional biomarkers for imminent birth selected from the group comprising ALDH1A1, CCL18, CCL2, CCL13, IL1RL1, CD14, CD163, TNFRSF8 or any combination thereof. Advantageously, the combination of biomarkers for preterm delivery before 32 weeks of gestation may further comprise one or more additional biomarkers for imminent birth selected from the group comprising ALDH1A1, CCL18, CCL2, CCL13, IL1RL1, CD14, CD163, TNFRSF8 or any combination thereof.

For example, at least three different biomarkers, advantageously at least four different biomarkers, advantageously at least five different biomarkers, advantageously at least six different biomarkers, advantageously at least seven different biomarkers, advantageously at least eight different biomarkers, advantageously at least nine different biomarkers, advantageously at least ten different biomarkers, up to and including all of these biomarkers may be used according to the present invention. All the combination of biomarkers which have been described above may be used according to the present invention.

In particularly advantageous embodiment, the present invention relates to the use of a combination of biomarkers for preterm delivery comprising COL1A1, CCN5, ALDH1A1, CCL18, CCL2, CCL13, IL1RL1, CD14, CD163 and TNFRSF8. Advantageously, the combination of biomarkers for its use in preterm delivery comprises COL1A1, CCN5, ALDH1A1, CCL18, CCL2, CCL13, IL1RL1, CD14, CD163 and TNFRSF8. Advantageously, the combination of biomarkers for its use in preterm delivery solely comprises COL1A1, CCN5, ALDH1A1, CCL18, CCL2, CCL13, IL1RL1, CD14, CD163 and TNFRSF8. Advantageously, the combination of biomarkers for its use in preterm delivery contains COL1A1, CCN5, ALDH1A1, CCL18, CCL2, CCL13, IL1RL1, CD14, CD163 and TNFRSF8.

In particularly advantageous embodiment, the present invention relates to the use of a combination of biomarkers for prognosing and/or for diagnosing, whether an individual is at risk of preterm delivery comprising COL1A1, CCN5, ALDH1A1, CCL18, CCL2, CCL13, IL1RL1, CD14, CD163 and TNFRSF8. Advantageously, the combination of biomarkers for its use in prognosing and/or for diagnosing, whether an individual is at risk of preterm delivery comprises COL1A1, CCN5, ALDH1A1, CCL18, CCL2, CCL13, IL1RL1, CD14, CD163 and TNFRSF8. Advantageously, the combination of biomarkers for its use in prognosing and/or for diagnosing, whether an individual is at risk of preterm delivery solely comprises COL1A1, CCN5, ALDH1A1, CCL18, CCL2, CCL13, IL1RL1, CD14, CD163 and TNFRSF8. Advantageously, the combination of biomarkers for its use in prognosing and/or for diagnosing, whether an individual is at risk of preterm delivery contains COL1A1, CCN5, ALDH1A1, CCL18, CCL2, CCL13, IL1RL1, CD14, CD163 and TNFRSF8.

In particularly advantageous embodiment, the present invention relates to the use of a combination of biomarkers for preterm delivery before 32 weeks of gestation comprising COL1A1, CCN5, ALDH1A1, CCL18, CCL2, CCL13, IL1RL1, CD14, CD163 and TNFRSF8. Advantageously, the combination of biomarkers for its use in preterm delivery before 32 weeks of gestation comprises COL1A1, CCN5, ALDH1A1, CCL18, CCL2, CCL13, IL1RL1, CD14, CD163 and TNFRSF8.

Advantageously, the combination of biomarkers for its use in preterm delivery before 32 weeks of gestation solely comprises COL1A1, CCN5, ALDH1A1, CCL18, CCL2, CCL13, IL1RL1, CD14, CD163 and TNFRSF8. Advantageously, the combination of biomarkers for its use in preterm delivery before 32 weeks of gestation contains COL1A1, CCN5, ALDH1A1, CCL18, CCL2, CCL13, IL1RL1, CD14, CD163 and TNFRSF8.

In particularly advantageous embodiment, the present invention relates to the use of a combination of biomarkers for prognosing and/or for diagnosing, whether an individual is at risk of preterm delivery before 32 weeks of gestation comprising COL1A1, CCN5, ALDH1A1, CCL18, CCL2, CCL13, IL1RL1, CD14, CD163 and TNFRSF8. Advantageously, the combination of biomarkers for its use in prognosing and/or for diagnosing, whether an individual is at risk of preterm delivery before 32 weeks of gestation comprises COL1A1, CCN5, ALDH1A1, CCL18, CCL2, CCL13, IL1RL1, CD14, CD163 and TNFRSF8. Advantageously, the combination of biomarkers for its use in prognosing and/or for diagnosing, whether an individual is at risk of preterm delivery before 32 weeks of gestation solely comprises COL1A1, CCN5, ALDH1A1, CCL18, CCL2, CCL13, IL1RL1, CD14, CD163 and TNFRSF8. Advantageously, the combination of biomarkers for its use in prognosing and/or for diagnosing, whether an individual is at risk of preterm delivery before 32 weeks of gestation contains COL1A1, CCN5, ALDH1A1, CCL18, CCL2, CCL13, IL1RL1, CD14, CD163 and TNFRSF8.

In particularly advantageous embodiment, the present invention relates to the use of a combination of biomarkers for imminent birth comprising COL1A1, CCN5, CCL18, CCL2, IL1RL1, CD14, CD163 and TNFRSF8. Advantageously, the combination of biomarkers for its use in imminent birth comprises COL1A1, CCN5, CCL18, CCL2, IL1RL1, CD14, CD163 and TNFRSF8. Advantageously, the combination of biomarkers for its use in imminent birth solely comprises COL1A1, CCN5, CCL18, CCL2, IL1RL1, CD14, CD163 and TNFRSF8. Advantageously, the combination of biomarkers for its use in imminent birth contains COL1A1, CCN5, CCL18, CCL2, IL1RL1, CD14, CD163 and TNFRSF8.

In particularly advantageous embodiment, the present invention relates to the use of a combination of biomarkers for prognosing and/or for diagnosing, whether an individual is at risk of imminent birth comprising COL1A1, CCN5, CCL18, CCL2, IL1RL1, CD14, CD163 and TNFRSF8. Advantageously, the combination of biomarkers for its use in prognosing and/or for diagnosing, whether an individual is at risk of imminent birth comprises COL1A1, CCN5, CCL18, CCL2, IL1RL1, CD14, CD163 and TNFRSF8. Advantageously, the combination of biomarkers for its use in prognosing and/or for diagnosing, whether an individual is at risk of imminent birth solely comprises COL1A1, CCN5, CCL18, CCL2, IL1RL1, CD14, CD163 and TNFRSF8. Advantageously, the combination of biomarkers for its use in prognosing and/or for diagnosing, whether an individual is at risk of imminent birth contains COL1A1, CCN5, CCL18, CCL2, IL1RL1, CD14, CD163 and TNFRSF8.

According to the present invention, the biomarker of the invention may be a protein selected from the group comprising COL1A1, CCN5, ALDH1A1, CCL18, CCL2, CCL13, IL1RL1, CD14, CD163, TNFRSF8 or a nucleic acid encoding for one of said proteins.

The present invention also provides methods for identifying individual that are at risk for preterm delivery, advantageously the risk for preterm delivery before 32 weeks of gestation and/or imminent birth. The combination of biomarkers as disclosed above has been identified that may be utilized to identify individuals during early to mid-pregnancy that may be at risk for preterm delivery, advantageously at risk for preterm delivery before 32 weeks of gestation and/or imminent birth. Such combination of biomarkers may allow the diagnostic distinction between preterm delivery and/or imminent birth and other conditions that exhibit similar symptoms. Such combination of biomarkers may also allow the prediction of preterm delivery and/or the prediction of imminent spontaneous delivery. Early identification of individuals at greater risk for preterm delivery and/or imminent birth would be of considerable value, as such subjects could be more closely monitored.

Advantageously, the present invention provides a method for prognosing and/or for diagnosing, whether an individual is at risk of preterm delivery and/or imminent birth, the method comprising:
a) determining the presence and/or amount of a combination of biomarkers for preterm delivery in a sample obtained from said individual, and
b) comparing the presence and/or amount of said combination of biomarkers obtained from step a) to the presence and/or amount of the same combination of biomarkers in a control sample, to identify an increased risk for preterm delivery,
wherein said combination of biomarkers comprises COL1A1 and CCN5.

As used herein, the term “diagnostic” or “diagnosing” refers to the procedure through which the presence of absence of a condition is identified in an individual. Advantageously, the condition is the preterm delivery. In one particular embodiment, the condition is the preterm delivery before 32 weeks of gestation. In one particular embodiment, the condition is imminent birth.

As used herein, the term “prognosis” or “prognosing” refers to the procedure through which a prediction is made of the events that will occur in the development or course of a condition. Advantageously, the condition is the preterm delivery. In one particular embodiment, the condition is the preterm delivery before 32 weeks of gestation. In one particular embodiment, the condition is imminent birth.

Advantageously, the inventors have shown that the method according to the invention makes it possible to exclude preterm delivery and thus exclude the risk of preterm delivery, when none or only one of the biomarkers are detected. In other words, the method according to the invention makes it possible to conclude with increased reliability on the absence of threat of preterm delivery, thus making it possible to avoid unnecessary hospitalizations. Advantageously, the inventors have shown that the method according to the invention makes it possible to exclude imminent birth and thus exclude the risk of preterm delivery, when none or only one of the biomarkers are detected. In other words, the method according to the invention makes it possible to conclude with increased reliability on the absence of threat of imminent birth, thus making it possible to avoid unnecessary hospitalizations.

In one aspect, prior to step a), the method may further comprises a step of obtaining a sample from an individual. Advantageously, the sample used in step a) is a sample of vaginal fluid, advantageously a sample of vaginal secretion. The sample that is taken from the individual may vary, but the sampling preferably is minimally invasive and is easily performed by conventional techniques.

Advantageously, the individual is pregnant individual. Advantageously, methods disclosed herein can be used to determine the risk or likelihood of preterm delivery in asymptomatic or symptomatic pregnant individuals. Advantageously, methods disclosed herein can be used to determine the risk or likelihood of preterm delivery before 32 weeks of gestation in asymptomatic or symptomatic pregnant individuals. Advantageously, methods disclosed herein can be used to determine the risk or likelihood of imminent birth in asymptomatic or symptomatic pregnant individuals. In particular embodiments, the pregnant individual is asymptomatic.

Testing of individual using the methods described herein may occur at any time during pregnancy, when the combination of biomarkers indicative of preterm delivery and/or imminent birth is quantifiable in the individual.

In one embodiment, the combination of biomarkers may be tested at 20 weeks of gestation, advantageously at 21 weeks of gestation, advantageously at 22 weeks of gestation, advantageously at 23 weeks of gestation, advantageously at 24 weeks of gestation, advantageously at 25 weeks of gestation, advantageously at 26 weeks of gestation, advantageously at 27 weeks of gestation, advantageously at 28 weeks of gestation, advantageously at 29 weeks of gestation, advantageously at 30 weeks of gestation, advantageously at 31 weeks of gestation, advantageously at 32 weeks of gestation, advantageously at 33 weeks of gestation, advantageously at 34 weeks of gestation, advantageously at 35 weeks of gestation, advantageously at 36 weeks of gestation, advantageously at 37 weeks of gestation.

In another embodiment, the combination of biomarkers may be tested at from about 20 weeks to about 37 weeks of gestation. Advantageously, the combination of biomarkers may be tested at from about 24 weeks to about 34 weeks of gestation. Advantageously, the combination of biomarkers may be tested at from about 28 weeks to about 32 weeks of gestation. It should be noted that these ranges should not be seen as limiting; as such testing may be performed at any point during pregnancy. Rather these ranges are provided to demonstrate periods of the gestational cycle, where such testing is most likely to occur in a majority of individuals.

Next, the method comprises determining the presence and/or amount of a combination of biomarkers for preterm delivery in a sample obtained from said individual, wherein said combination of biomarkers comprises COL1A1 and CCN5.

Advantageously, step a) of the method consists of determining the presence and/or amount of COL1A1 and CCN5 biomarkers in a sample obtained from an individual. Advantageously, the method of the invention makes it possible to predict preterm delivery and/or imminent birth and thus a high risk of preterm delivery and/or imminent birth, if COL1A1 and CCN5 biomarkers are present in the sample obtained from an individual. COL1A1 and CCN5 may be used in methods for prognosing and/or diagnosing individuals at risk of preterm delivery and/or imminent birth, and methods for diagnosing whether an individual is at risk of preterm delivery and/or imminent birth, or for prognosing whether an individual is at risk of preterm delivery and/or imminent birth. Advantageously, COL1A1 and CCN5 may be used in methods for prognosing and/or diagnosing individuals at risk of preterm delivery before 32 weeks of gestation, and methods for diagnosing whether an individual is at risk of preterm delivery before 32 weeks of gestation, or for prognosing whether an individual is at risk of preterm delivery before 32 weeks of gestation. Advantageously, COL1A1 and CCN5 may be used in methods for prognosing and/or diagnosing individuals at risk of imminent birth, and methods for diagnosing whether an individual is at risk of imminent birth, or for prognosing whether an individual is at risk of imminent birth. Variation of the amount of the biomarker, as compared to a control or reference amount, may indicate that the individual is at risk of preterm delivery and/or imminent birth. Presence or absence of the biomarker, as compared to a control or reference, may indicate that the individual is at risk of preterm delivery and/or imminent birth. Such methods involve determining the presence and/or the amount of COL1A1 and CCN5 in a sample obtained from the individual being tested. In some aspects, the methods involve determining the presence and/or the amount of all of the biomarkers COL1A1 and CCN5, and prognosing the risk of preterm delivery, and advantageously the risk of preterm delivery before 32 weeks of gestation and/or imminent birth. Advantageously, the method of the invention makes it possible to predict preterm delivery and thus a high risk of preterm delivery and/or imminent birth, if the amount of COL1A1 is higher than 3657 pg/ml and the amount of CCN5 is higher than 4714 pg/ml in the sample obtained from an individual. Advantageously, if in a sample the amount of COL1A1 is higher than 3657 pg/ml and the amount of CCN5 is higher than 4714 pg/ml, the individual presents a risk of at least 75%, advantageously at least 80%, advantageously at least 85%, advantageously at least 90%, advantageously at least 95%, advantageously at least 96%, advantageously at least 97%, advantageously at least 98%, advantageously at least 99%, advantageously at least 100% of preterm delivery.

Advantageously, if in a sample the amount of COL1A1 is higher than 3657 pg/ml and the amount of CCN5 is higher than 4714 pg/ml, the individual presents a risk of at least 75%, advantageously at least 80%, advantageously at least 85%, advantageously at least 90%, advantageously at least 95%, advantageously at least 96%, advantageously at least 97%, advantageously at least 98%, advantageously at least 99%, advantageously at least 100% of preterm delivery before 32 weeks of gestation.

Advantageously, if in a sample the amount of COL1A1 is higher than 3657 pg/ml and the amount of CCN5 is higher than 4714 pg/ml, the individual presents a risk of at least 75%, advantageously at least 80%, advantageously at least 85%, advantageously at least 90%, advantageously at least 95%, advantageously at least 96%, advantageously at least 97%, advantageously at least 98%, advantageously at least 99%, advantageously at least 100% of imminent birth.

In one embodiment, the combination of biomarkers used in step a) may further comprises one or more additional biomarkers for preterm delivery selected from the group comprising ALDH1A1, CCL18, CCL2, CCL13, IL1RL1, CD14, CD163, TNFRSF8 or any combination thereof. Advantageously, the method of the invention makes it possible to predict preterm delivery and thus a high risk of preterm delivery, advantageously a high risk of preterm delivery before 32 weeks of gestation, if COL1A1 and CCN5 biomarkers and one or more additional biomarkers are present in the sample obtained from an individual. Advantageously, the method of the invention makes it possible to predict imminent birth and thus a high risk of imminent birth, if COL1A1 and CCN5 biomarkers and one or more additional biomarkers are present in the sample obtained from an individual.

Each of the additional biomarkers ALDH1A1, CCL18, CCL2, CCL13, IL1RL1, CD14, CD163 and TNFRSF8 may be used in methods for prognosing and/or diagnosing individuals at risk of preterm delivery and/or imminent birth, and methods for diagnosing whether an individual is at risk of preterm delivery and/or imminent birth, or for prognosing whether an individual is at risk of preterm delivery and/or imminent birth. Advantageously, ALDH1A1, CCL18, CCL2, CCL13, IL1RL1, CD14, CD163 and TNFRSF8 may be used in methods for prognosing and/or diagnosing individuals at risk of preterm delivery before 32 weeks of gestation, and methods for diagnosing whether an individual is at risk of preterm delivery before 32 weeks of gestation, or for prognosing whether an individual is at risk of preterm delivery before 32 weeks of gestation.

Advantageously, ALDH1A1, CCL18, CCL2, CCL13, IL1RL1, CD14, CD163 and TNFRSF8 may be used in methods for prognosing and/or diagnosing individuals at risk of imminent birth, and methods for diagnosing whether an individual is at risk of imminent birth, or for prognosing whether an individual is at risk of imminent birth.

Variation of the amount of the additional biomarker, as compared to a control or reference amount, may indicate that the individual is at risk of preterm delivery and/or imminent birth. Presence or absence of the additional biomarker, as compared to a control or reference, may indicate that the individual is at risk of preterm delivery, advantageously if the individual is at risk of preterm delivery before 32 weeks of gestation and/or imminent birth. Such methods involve determining the presence and/or the amount of ALDH1A1, CCL18, CCL2, CCL13, IL1RL1, CD14, CD163 and TNFRSF8 in a sample obtained from the individual being tested. In some aspects, the methods involve determining the presence and/or the amount of all of the additional biomarkers ALDH1A1, CCL18, CCL2, CCL13, IL1RL1, CD14, CD163 and TNFRSF8, and prognosing the risk of preterm delivery, advantageously the risk of preterm delivery before 32 weeks of gestation and/or imminent birth.

Advantageously, the method of the invention makes it possible to predict preterm delivery and thus a high risk of preterm delivery, advantageously a risk of preterm delivery before 32 weeks of gestation, if the respective amount of COL1A and CCN5 is as follows:
amount of COL1A1 is higher than 3657,000 pg/ml,
amount of CCN5 is higher than 4714,000 pg/ml, and the amount of one or more additional biomarkers is as follows:
amount of ALDH1A1 is higher than 1171,000 pg/ml, and/or
amount of CCL18 is higher than 327,300 pg/ml, and/or
amount of CCL2 is higher than 93,410 pg/ml, and/or
amount of CCL13 is higher than 3,615 pg/ml, and/or
amount of IL1RL1 is higher than 9319,000 pg/ml, and/or
amount of CD14 is higher than 16514,000 pg/ml, and/or
amount of CD163 is higher than 12791,000 pg/ml, and/or
amount of TNFRSF8 is higher than 3,355 pg/ml.

In a particularly advantageous embodiment, the combination of biomarkers for preterm delivery used in step a) comprises COL1A1, CCN5, ALDH1A1, CCL18, CCL2, CCL13, IL1RL1, CD14, CD163 and TNFRSF8. Advantageously, the method of the invention makes it possible to predict preterm delivery and thus a high risk of preterm delivery, advantageously a risk of preterm delivery before 32 weeks of gestation, if COL1A1, CCN5, ALDH1A1, CCL18, CCL2, CCL13, IL1RL1, CD14, CD163 and TNFRSF8 biomarkers are present in the sample obtained from an individual.

Advantageously, the method of the invention makes it possible to predict preterm delivery and thus a high risk of preterm delivery, advantageously a risk of preterm delivery before 32 weeks of gestation, if the respective amount of each biomarkers of the combination of biomarkers in the sample obtained from an individual is as follows:
amount of COL1A1 is higher than 3657,000 pg/ml, and
amount of CCN5 is higher than 4714,000 pg/ml, and
amount of ALDH1A1 is higher than 1171,000 pg/ml, and
amount of CCL18 is higher than 327,300 pg/ml, and
amount of CCL2 is higher than 93,410 pg/ml, and
amount of CCL13 is higher than 3,615 pg/ml, and
amount of IL1RL1 is higher than 9319,000 pg/ml, and
amount of CD14 is higher than 16514,000 pg/ml, and
amount of CD163 is higher than 12791,000 pg/ml, and
amount of TNFRSF8 is higher than 3,355 pg/ml.

Advantageously, if in a sample the respective amount of each biomarkers of the combination of biomarkers is higher than the amount described above, the individual presents a risk of at least 75%, advantageously at least 80%, advantageously at least 85%, advantageously at least 90%, advantageously at least 95%, advantageously at least 96%, advantageously at least 97%, advantageously at least 98%, advantageously at least 99%, advantageously at least 100% of preterm delivery. Advantageously, if in a sample the respective amount of each biomarkers of the combination of biomarkers is higher than the amount described above, the individual presents a risk of at least 75%, advantageously at least 80%, advantageously at least 85%, advantageously at least 90%, advantageously at least 95%, advantageously at least 96%, advantageously at least 97%, advantageously at least 98%, advantageously at least 99%, advantageously at least 100% of preterm delivery before 32 weeks of gestation

Advantageously, the method of the invention makes it possible to predict imminent birth and thus a high risk of imminent birth, if the respective amount of COL1A and CCN5 is as follows:
amount of COL1A1 is higher than 3657,000 pg/ml,
amount of CCN5 is higher than 4714,000 pg/ml, and the amount of one or more additional biomarkers is as follows:
amount of CCL18 is higher than 327,300 pg/ml, and/or
amount of CCL2 is higher than 93,410 pg/ml, and/or
amount of IL1RL1 is higher than 9319,000 pg/ml, and/or
amount of CD14 is higher than 16514,000 pg/ml, and/or
amount of CD163 is higher than 12791,000 pg/ml, and/or
amount of TNFRSF8 is higher than 3,355 pg/ml.

In a particularly advantageous embodiment, the combination of biomarkers for preterm delivery used in step a) comprises COL1A1, CCN5, ALDH1A1, CCL18, CCL2, CCL13, IL1RL1, CD14, CD163 and TNFRSF8. Advantageously, the method of the invention makes it possible to predict imminent birth and thus a high risk of imminent birth, if COL1A1, CCN5, CCL18, CCL2, IL1RL1, CD14, CD163 and TNFRSF8 biomarkers are present in the sample obtained from an individual.

Advantageously, the method of the invention makes it possible to predict imminent birth and thus a high risk of imminent birth, if the respective amount of each biomarkers of the combination of biomarkers in the sample obtained from an individual is as follows:
amount of COL1A1 is higher than 3657,000 pg/ml, and
amount of CCN5 is higher than 4714,000 pg/ml, and
amount of CCL18 is higher than 327,300 pg/ml, and
amount of CCL2 is higher than 93,410 pg/ml, and
amount of IL1RL1 is higher than 9319,000 pg/ml, and
amount of CD14 is higher than 16514,000 pg/ml, and
amount of CD163 is higher than 12791,000 pg/ml, and
amount of TNFRSF8 is higher than 3,355 pg/ml.

Advantageously, if in a sample the respective amount of each biomarkers of the combination of biomarkers is higher than the amount described above, the individual presents a risk of at least 75%, advantageously at least 80%, advantageously at least 85%, advantageously at least 90%, advantageously at least 95%, advantageously at least 96%, advantageously at least 97%, advantageously at least 98%, advantageously at least 99%, advantageously at least 100% of imminent birth.

Measurements of biomarker may include, for example, measurements that indicate the presence, amount, expression level, or any other value associated with a biomarker. The biomarkers of the invention may be detected at the nucleic acid or protein level. Thus, the biomarkers of the invention may be nucleic acid encoding for a protein such as DNA, RNA or a protein and may be detected using any appropriate technique. The presence and/or amount of the combination of biomarkers of the invention may be measured directly or indirectly. Any appropriate agent may be used to determine the presence and/or amount of the one or more biomarker of the invention. For example, the presence and/or amount of the combination of biomarkers of the invention may be determined using an agent selected from peptides and peptidomimetics, antibodies, small molecules and oligonucleotides such as single-stranded DNA or RNA molecules, as described herein. The relative presence and/or amount of the combination of biomarkers of the invention relative to a control sample may be determined using any appropriate technique. Suitable standard techniques are known in the art.

For example, when the biomarkers of the combination are detected at the nucleic acid level this may be carried out using: (i) biomarker-specific oligonucleotide DNA or RNA or any other nucleic acid derivative probes bound to a solid surface; (ii) purified RNA (labelled by any method, for example using reverse transcription and amplification) hybridized to probes; (iii) purified RNA hybridized to probes and a second probe (labelled by any method) hybridized to the purified RNA; (iv) RT-PCR using any primer/probe combination or inter-chelating fluorescent label; (v) end-point PCR; (vi) digital PCT; (vii) sequencing; (viii) array cards (RT-PCT); (ix) lateral flow devices/methodology; and/or (x) digital microfluidics.

For example, when the biomarkers of the combination are detected at the protein acid level this may be carried out using: (i) biomarker-specific primary antibodies or antibody fragments bound to a solid surface; (ii) secondary biomarker-specific antibodies or antibody fragments used to detect biomarker antigen bound to primary antibody (labelled using any method); (iii) biomarker-specific primary aptamers bound to a solid surface; (iv) secondary biomarker-specific aptamer used to detect biomarker antigen bound to primary aptamer (labelled using any method); (v) any antibody derivative i.e. phage display etc. used as above; (vi) lateral flow devices/methodology; (vii) chromatography; (viii) mass spectrometry; (ix) nuclear magnetic resonance (MR); (x) protein gels/transfers to filter; and/or (xi) immunoprecipitation.

The presence and/or amount of the biomarkers of the combination of the invention may be determined by quantitative and/or qualitative analysis. The amount of the biomarkers of the combination of the invention encompasses the mass of the biomarkers of the combination, the molar amount of the biomarkers of the combination, the concentration of the biomarkers of the combination and the molarity of the biomarkers of the combination. This amount may be given in any appropriate units. For example, the concentration of the biomarkers of the combination may be given in pg/ml, ng/ml, pg/ml or mg/ml.

Any agent for the detection of or for the determination of the amount of the biomarkers of the combination of the invention may be used to determine the presence of and/or amount of the biomarkers of the combination. Similarly, any method that allows for the diagnosing and/or prognosing of the biomarkers of the combination, the quantification, or relative quantification of the biomarkers of the combination may be used.

Agents for the detection of or for the determination of the amount of one biomarker present in the combination or several biomarkers of the combination may be used to determine the amount of the one biomarker present in the combination or several biomarkers of the combination in a sample obtained from the individual. Such agents typically bind to one biomarker present in the combination or several biomarkers of the combination. Such agents may bind specifically to one biomarker present in the combination or several biomarkers of the combination. The agent for the detection of or for the determination of the amount of the biomarkers of the combination may be an antibody or other binding agent specific for one biomarker present in the combination or several biomarkers of the combination. By specific, it will be understood that the agent or antibody binds to the molecule of interest, in this case one or more biomarkers of the combination, with no significant cross-reactivity to any other molecule, particularly any other protein. Cross-reactivity may be assessed by any suitable method. Cross-reactivity of an agent or antibody for one or more biomarkers of the combination with a molecule other than one or more biomarkers of the combination may be considered significant if the agent or antibody binds to the other molecule at least 5%, 10%, 15%, 20%, 25%, 30%, 35%, 40%, 45%, 50%, 55%, 60%, 65%, 70%, 75%, 80%, 85%, 90% or 100% as strongly as it binds to one or more biomarkers of the combination. An agent or antibody that is specific for one or more biomarkers of the combination may bind to another molecule at less than 90%, 85%, 80%, 75%, 70%, 65%, 60%, 55%, 50%, 45%, 40%, 35%, 30%, 25% or 20%) the strength that it binds to one or more biomarkers of the combination. Advantageously, the agent or antibody binds to the other molecule at less than 20%, less than 15%, less than 10% or less than 5%), less than 2% or less than 1% the strength that it binds to one or more biomarkers of the combination.

In one embodiment, in step a), the presence and/or amount of the COL1A1 and CCN5 biomarkers is determined using an antibody specific for said COL1A1 and CCN5 biomarkers. Advantageously, when the combination of COL1A1 and CCN5 biomarkers comprises one or more additional biomarkers, the presence and/or amount of the one or more additional biomarkers is determined using an antibody specific for said one or more additional biomarkers.

As described herein, the presence and/or amount of the combination of biomarkers may be determined immunologically by reacting antibodies, or functional fragments thereof, specific to the biomarkers. A functional fragment of an antibody is a portion of an antibody that retains at least some ability to bind to the antigen to which the complete antibody binds. The fragments, which include, but are not limited to, scFv fragments, Fab fragments, F(ab) fragments and F(ab)2 fragments, can be recombinantly produced or enzymatically produced. Specific binding molecules other than antibodies, such as aptamers, may be used to bind the biomarkers.

The antibody may be monoclonal or polyclonal. The antibody may be produced by any suitable method known in the art. For example, polyclonal antibodies may be obtained by immunizing a mammal, typically a rabbit or a mouse, with the one or more biomarker under suitable conditions and isolating antibody molecules from, for example, the serum of said mammal. Monoclonal antibodies may be obtained by hybridoma or recombinant methods. Hybridoma methods may involve immunizing a mammal, typically a rabbit or a mouse, with the one or more biomarker under suitable conditions, then harvesting the spleen cells of said mammal and fusing them with myeloma cells. The mixture of fused cells is then diluted and clones are grown from single parent cells. The antibodies secreted by the different clones are then tested for their ability to bind to the one or more biomarker, and the most productive and stable clone is then grown in culture medium to a high volume. The secreted antibody is collected and purified.

Recombinant methods may involve the cloning into phage or yeast of different immunoglobulin gene segments to create libraries of antibodies with slightly different amino acid sequences. Those sequences which give rise to antibodies which bind to the one or more biomarker may be selected and the sequences cloned into, for example, a bacterial cell line, for production. Typically the antibody is a mammalian antibody, such as a primate, human, rodent (e.g. mouse or rat), rabbit, ovine, porcine, equine or camel antibody. The antibody may be a camelid antibody or shark antibody. The antibody may be a nanobody. The antibody can be any class or isotype of antibody, for example IgM, but is preferably IgG. The antibody may be a humanized antibody.

The antibody or fragment may be associated with other moieties, such as linkers which may be used to join together 2 or more fragments or antibodies. Such linkers may be chemical linkers or can be present in the form of a fusion protein with the fragment or whole antibody. The linkers may thus be used to join together whole antibodies or fragments which have the same or different binding specificities, e.g. that can bind the same or different polymorphisms. The antibody may be a bispecific antibody which is able to bind to two different antigens, typically any two of the polymorphisms mentioned herein. The antibody may be a ‘diabody’ formed by joining two variable domains back to back. In the case where the antibodies used in the method are present in any of the above forms which have different antigen binding sites of different specificities then these different specificities are typically to polymorphisms at different positions or on different proteins. In one embodiment the antibody is a chimeric antibody comprising sequence from different natural antibodies, for example a humanized antibody.

Methods to assess an amount of the biomarkers of the combination may involve contacting a sample with an agent or antibody capable of binding specifically to the biomarkers of the combination. Such methods may include dipstick assays and Enzyme-linked Immunosorbant Assay (ELISA), or similar assays, such as those using a lateral flow device. Other immunoassay types may also be used to assess the one or more biomarker amounts. Typically dipsticks comprise one or more antibodies or proteins that specifically bind to the biomarkers of the combination. If more than one antibody is present, the antibodies preferably have different non-overlapping determinants such that they may bind to the biomarkers of the combination simultaneously.

ELISA is a heterogeneous, solid phase assay that requires the separation of reagents. ELISA is typically carried out using the sandwich technique or the competitive technique. The sandwich technique requires two antibodies. The first specifically binds one or more biomarkers of the combination and is bound to a solid support. The second antibody is bound to a marker, typically an enzyme conjugate. A substrate for the enzyme is used to quantify biomarkers of the combination-antibody complex and hence the amount of the biomarkers of the combination in a sample. The antigen competitive inhibition assay also typically requires that the biomarkers of the combination-specific antibody bound to a support. A biomarker-enzyme conjugate is added to the sample containing one or more biomarkers of the combination to be assayed. Competitive inhibition between the biomarker-enzyme conjugate and unlabelled biomarker allows quantification of the amount of the biomarkers of the combination in a sample. The solid supports for ELISA reactions preferably contain wells.

Multiplex approach such as antibodies on Luminex beads (for example of the multiplex bio-rad), the approach singleplex or multiplex by chemiluminescence of the company MesoScale Discovery (MSD), or antibodies on flat surface (“Antibodies-Arrays”) (for example the approach proposed by the company MesoScale Discovery) may also be used for determining the presence and/or amount of the combination of biomarkers of the invention.

Antibodies capable of binding specifically to the biomarkers of the combination may be used in methods of immunofluorescence to detect the presence of the biomarkers of the combination and hence in methods of diagnosing and/or prognosing the risk of preterm delivery, advantageously the risk of preterm delivery before 32 weeks of gestation, and/or imminent birth according to the present invention.

The present invention may also employ methods of determining the amount of the biomarkers of the combination that do not comprise antibodies. High Performance Liquid Chromatography (HPLC) separation and fluorescence detection is preferably used as a method of determining the amount of the biomarkers of the combination.

Other methods of determining the amount the biomarkers of the combination that do not comprise antibodies include mass spectrometry. Mass spectrometric methods may include, for example, matrix-assisted laser desorption/ionization mass spectrometry (MALDI MS), surface-enhanced laser desorption/ionization mass spectrometry (SELDI MS), time of flight mass spectrometry (TOF MS) and liquid chromatography mass spectrometry (LC MS). in that case, prior to the measurements, a fixed amount of a least one substance serving as the at least on internal standard is added to the original sample and the intensity of its peak is also measured. The concentration of the target in the sample can be calculated from the ratio of peak intensity of the target to the peak intensity of the at least one internal standard.

A separation method may be used to determine the presence and/or amount of the biomarkers of the combination, such that only a subset of biomarkers within the sample is analyzed. For example, the biomarkers that are analyzed in a sample may consist of mRNA species from a cellular extract, which has been fractionated to obtain only the nucleic acid biomarkers within the sample, or the biomarkers may consist of a fraction of the total complement of proteins within the sample, which have been fractionated by chromatographic techniques. One or more, two or more, three or more, four or more, or five or more separation methods may be used according to the present invention.

Determination of the presence and/or amount of the biomarkers of the combination may be carried out without employing a separation method. For example, a biological sample may be interrogated with a labelled compound that forms a specific complex with a biomarker in the sample, where the intensity of the label in the specific complex is a measurable characteristic of the biomarker. A suitable compound for forming such a specific complex is a labelled antibody. A biomarker may be measured using an antibody with an amplifiable nucleic acid as a label. The nucleic acid label may become amplifiable when two antibodies, each conjugated to one strand of a nucleic acid label, interact with the biomarker, such that the two nucleic acid strands form an amplifiable nucleic acid.

The presence and/or amount of the biomarkers of the combination may be derived from an assay, such as an array, of nucleic acids, where the biomarkers are the nucleic acids or complements thereof. For example, the biomarkers may be nucleic acids. The presence and/or amount of the biomarkers of the combination may be obtained using a method selected from nuclear magnetic resonance, nucleic acid arrays, dot blotting, slot blotting, reverse transcription amplification, Northern analysis and quantitative real-time PCR (qPCR).

The determination of the presence and/or amount of the biomarkers of the combination may be generated by the use of one or more separation methods. For example, suitable separation methods may include a mass spectrometry method, such as electrospray ionization mass spectrometry (ESI-MS), ESI-MS/MS, ESI-MS/(MS)n (n is an integer greater than zero), matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SLMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n. Other mass spectrometry methods may include, inter alia, quadrupole, Fourier transform mass spectrometry (FTMS) and ion trap. Other suitable separation methods may include chemical extraction partitioning, column chromatography, ion exchange chromatography, hydrophobic (reverse phase) liquid chromatography, isoelectric focusing, one-dimensional polyacrylamide gel electrophoresis (PAGE), two-dimensional polyacrylamide gel electrophoresis (2D-PAGE) or other chromatography, such as thin-layer, gas or liquid chromatography, or any combination thereof. The sample may be fractionated prior to application of the separation method.

The determination of the presence and/or amount of the biomarkers of the combination may be generated by methods that do not require physical separation of the biomarkers themselves. For example, nuclear magnetic resonance (MR) spectroscopy may be used to resolve a profile of biomarkers from a complex mixture of molecules.

In another embodiment, the total mRNA from a cellular extract of the individual is assayed, and the various mRNA species that are obtained from the sample are used as biomarkers. Biomarker may be obtained, for example, by hybridizing these mRNAs to an array of probes, which may comprise oligonucleotides or cDNAs, using standard methods known in the art. Alternatively, the mRNAs may be subjected to gel electrophoresis or blotting methods such as dot blots, slot blots or Northern analysis, all of which are known in the art. mRNA profiles also may be obtained by reverse transcription followed by amplification and detection of the resulting cDNAs. In another embodiment, the profile may be obtained by using a combination of methods, such as a nucleic acid array combined with mass spectroscopy.

Different methods have different advantages and may be preferred depending on numerous factors, such as the particular circumstances of the individuals to be tested and/or the availability of reagents/equipment in the diagnostics laboratory. For example, qPCR using probe/quencher hydrolysis probes as described herein is highly specific and stringent. As another example, microarray analysis can resolve subtle differences in expression of transcript variants, which may be important in disease pathology and diagnosis.

Any appropriate detection means can be used to detect or quantify the biomarkers of the combination of the invention, as described herein.

Typically, when the biomarkers of the combination of the invention are a nucleic acid, the presence of the biomarkers of the combination may be detected, and/or the amount of the biomarkers of the combination determined using an oligonucleotide probe.

In one embodiment, in step a), the presence and/or amount of the COL1A1 and CCN5 biomarkers is determined using an oligonucleotide probe specific for said COL1A1 and CCN5 biomarkers. Advantageously, when the combination of COL1A1 and CCN5 biomarkers comprises one or more additional biomarkers, the presence and/or amount of the one or more additional biomarkers is determined using an oligonucleotide probe specific for said one or more additional biomarkers.

An oligonucleotide probe of the invention may have at least 80% sequence identity to the biomarkers of the combination of the invention, or a target region within said biomarkers, measured over any appropriate length of sequence. Typically the % sequence identity is determined over a length of contiguous nucleic acid residues. An oligonucleotide probe of the invention may, for example, have at least 80% sequence identity to the biomarkers of the combination of the invention, or target region thereof, measured over at least at least 20, at least 30, at least 40, at least 50, at least 60, at least 70, at least 80, at least 90, or more nucleic acid residues, up to the oligonucleotide probe having at least 80%>sequence identity with the biomarkers of the combination of the invention, or target region thereof, over the entire length of the oligonucleotide probe.

An oligonucleotide probe of the invention may be complementary to the one or more nucleic acid biomarker of the invention, or a target region thereof. Typically the oligonucleotide probe of the invention is complementary over a length of contiguous nucleic acid residues. An oligonucleotide probe of the invention may, for example, be complementary to the biomarkers of the combination of the invention, or target region thereof, measured over at least 10, at least 20, at least 30, at least 40, at least 50, at least 60, at least 70, at least 80, at least 90, or more nucleic acid residues, up to the oligonucleotide probe having being complementary to the one or more biomarker of the invention, or target region thereof, over the entire length of the oligonucleotide probe.

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. The probes of the invention are typically designed to hybridize to their target nucleic acid sequence present in the biomarkers of the combination of the invention.

Typically, probes of the invention are oligonucleotides having sequence identity with a region of the biomarkers of the combination of the invention as disclosed herein. One or more probe may be immobilized on a solid support, and used to interrogate mRNA obtained from a test sample. If the mRNA from the test sample contains the one or more biomarker targeted by the immobilized probe, it will bind to the probe, and may then be detected. The biomarkers of the invention may also be detected using PCR, such as real time PCR. Any oligonucleotide with the appropriate level of sequence identity with the biomarkers of the combination of the invention, or with one or more target sequences within said biomarkers of the combination of the invention may be used as a probe as described herein. Any oligonucleotide with the appropriate level of complementarity with the one or more biomarker of the invention, or with one or more target sequences within said biomarkers of the combination of the invention may be used as a probe as described herein.

Next, the method comprises a step b) consisting of comparing the presence and/or amount of said combination of biomarkers obtained from step a) to the presence and/or amount of the same combination of biomarkers in a control sample, to identify an increased risk for preterm delivery, advantageously an increased risk for preterm delivery before 32 weeks of gestation and/or imminent birth.

As used herein, “comparing” or “comparison” includes any means to discern at least one difference in the presence and/or amount of the biomarkers of the combination in the individual and the control sample. Thus, a comparison may include a visual inspection of chromatographic spectra, and a comparison may include arithmetical or statistical comparisons of values assigned to the features of the profiles. Such statistical comparisons include, but are not limited to, applying a decision rule. The comparison can confirm the presence or absence of preterm delivery and/or imminent birth, and thus to prognose or diagnose a risk of preterm delivery and/or imminent birth. Advantageously, the comparison can confirm the presence or absence of preterm delivery, and thus to prognose or diagnose a risk of preterm delivery before 32 weeks of gestation.

Typically, the control sample corresponds to a sample of vaginal fluid, advantageously a sample of vaginal secretion obtained from individual that did not experience preterm delivery and/or imminent birth. In other words, the control sample corresponds to a sample of vaginal fluid obtained from individual, who had normal birth, also named control population.

Advantageously, in the control sample, the amount of the biomarkers is as follows:
amount of COL1A1 is less or equal to 3657,000 pg/ml, and
amount of CCN5 is less or equal to 4714,000 pg/ml, and
amount of ALDH1A1 is less or equal to 1171,000 pg/ml, and
amount of CCL18 is less or equal to 327,300 pg/ml, and
amount of CCL2 is less or equal to 93,410 pg/ml, and
amount of CCL13 is less or equal to 3,615 pg/ml, and
amount of IL1RL1 is less or equal to 9319,000 pg/ml, and
amount of CD14 is less or equal to 16514,000 pg/ml, and
amount of CD163 is less or equal to 12791,000 pg/ml, and
amount of TNFRSF8 is less or equal to 3,355 pg/ml.

In another embodiment, the method may further comprise comparing the presence and/or amount of the combination of biomarkers obtained from step a) to the presence and/or amount of the same combination of biomarkers in a control sample; and classifying the individual as belonging to or not belonging to the control population, wherein the comparison determines in increased risk for preterm delivery and/or imminent birth and wherein the combination of biomarkers comprises COL1A1 and CCN5.

In another embodiment, the method may further comprise comparing the presence and/or amount of the combination of biomarkers obtained from step a) to the presence and/or amount of the same combination of biomarkers in a control sample; and classifying the individual as belonging to or not belonging to the control population, wherein the comparison determines in increased risk for preterm delivery and/or imminent birth, and wherein the combination of biomarkers comprises COL1A1, CCN5 and one or more additional biomarkers selected among the group comprising ALDH1A1, CCL18, CCL2, CCL13, IL1RL1, CD14, CD163 and TNFRSF8.

In another embodiment, the method may further comprise comparing the presence and/or amount of the combination of biomarkers obtained from step a) to the presence and/or amount of the same combination of biomarkers in a control sample; and classifying the individual as belonging to or not belonging to the control population, wherein the comparison determines in increased risk for preterm delivery and/or imminent birth, and wherein the combination of biomarkers comprises COL1A1, CCN5, ALDH1A1, CCL18, CCL2, CCL13, IL1RL1, CD14, CD163 and TNFRSF8.

Advantageously, if the amounts of the biomarkers of the combination in the sample obtained from the individual are higher than thus observed in the control sample, the individual presents a risk of at least 75%, advantageously at least 80%, advantageously at least 85%, advantageously at least 90%, advantageously at least 95%, advantageously at least 96%, advantageously at least 97%, advantageously at least 98%, advantageously at least 99%, advantageously at least 100% of preterm delivery and/or imminent birth.

Advantageously, if the amounts of the biomarkers of the combination in the sample obtained from the individual are higher than thus observed in the control sample, the individual presents a risk of at least 75%, advantageously at least 80%, advantageously at least 85%, advantageously at least 90%, advantageously at least 95%, advantageously at least 96%, advantageously at least 97%, advantageously at least 98%, advantageously at least 99%, advantageously at least 100% of preterm delivery before 32 weeks of gestation.

In one embodiment, the present invention relates to a method for prognosing whether an individual is at risk of preterm delivery, the method comprising:
a) obtaining a sample of vaginal fluid from an individual,
b) determining the presence and/or amount of the combination of biomarkers using an antibody specific for the combination of biomarkers in the sample of step a), and
c) comparing the presence and/or amount of said combination of biomarkers obtained from step b) to the presence and/or amount of said combination of biomarkers in a control sample, to identify an increased risk for preterm delivery,
wherein the combination of biomarkers is COL1A1, CCN5, ALDH1A1, CCL18, CCL2, CCL13, IL1RL1, CD14, CD163 and TNFRSF8.

In one embodiment, the present invention relates to a method for prognosing whether an individual is at risk of preterm delivery before 32 weeks of gestation, the method comprising:
a) obtaining a sample of vaginal fluid from an individual,
b) determining the presence and/or amount of the combination of biomarkers using an antibody specific for the combination of biomarkers in the sample of step a), and
c) comparing the presence and/or amount of said combination of biomarkers obtained from step b) to the presence and/or amount of said combination of biomarkers in a control sample, to identify an increased risk for preterm delivery before 32 weeks of gestation,
wherein the combination of biomarkers is COL1A1, CCN5, ALDH1A1, CCL18, CCL2, CCL13, IL1RL1, CD14, CD163 and TNFRSF8.

In one embodiment, the present invention relates to a method for prognosing whether an individual is at risk of imminent birth, the method comprising:
a) obtaining a sample of vaginal fluid from an individual,
b) determining the presence and/or amount of the combination of biomarkers using an antibody specific for the combination of biomarkers in the sample of step a), and
c) comparing the presence and/or amount of said combination of biomarkers obtained from step b) to the presence and/or amount of said combination of biomarkers in a control sample, to identify an increased risk of imminent birth,
wherein the combination of biomarkers is COL1A1, CCN5, CCL18, CCL2, IL1RL1, CD14, CD163 and TNFRSF8.

In another embodiment, the present invention relates to a method for prognosing whether an individual is at risk of preterm delivery, the method comprising:
a) obtaining a sample of vaginal fluid from an individual,
b) determining the presence and/or amount of the combination of biomarkers using an oligonucleotide specific for the combination of biomarkers in the sample of step a), and
c) comparing the presence and/or amount of said combination of biomarkers obtained from step b) to the presence and/or amount of said combination of biomarkers in a control sample, to identify an increased risk for preterm delivery,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for prognosing whether an individual is at risk of preterm delivery before 32 weeks of gestation, the method comprising:
a) obtaining a sample of vaginal fluid from an individual,
b) determining the presence and/or amount of the combination of biomarkers using an oligonucleotide specific for the combination of biomarkers in the sample of step a), and
c) comparing the presence and/or amount of said combination of biomarkers obtained from step b) to the presence and/or amount of said combination of biomarkers in a control sample, to identify an increased risk for preterm delivery before 32 weeks of gestation,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for prognosing whether an individual is at risk of imminent birth, the method comprising:
a) obtaining a sample of vaginal fluid from an individual,
b) determining the presence and/or amount of the combination of biomarkers using an oligonucleotide specific for the combination of biomarkers in the sample of step a), and
c) comparing the presence and/or amount of said combination of biomarkers obtained from step b) to the presence and/or amount of said combination of biomarkers in a control sample, to identify an increased risk of imminent birth,
wherein the combination of biomarkers is COL1A1, CCN5, CCL18, CCL2, IL1RL1, CD14, CD163 and TNFRSF8.

In another embodiment, the present invention relates to a method for diagnosing whether an individual is at risk of preterm delivery, the method comprising:
a) obtaining a sample of vaginal fluid from an individual,
b) determining the presence and/or amount of the combination of biomarkers using an antibody specific for the combination of biomarkers in the sample of step a), and
c) comparing the presence and/or amount of said combination of biomarkers obtained from step b) to the presence and/or amount of said combination of biomarkers in a control sample, to identify an increased risk for preterm delivery,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for diagnosing whether an individual is at risk of preterm delivery before 32 weeks of gestation, the method comprising:
a) obtaining a sample of vaginal fluid from an individual,
b) determining the presence and/or amount of the combination of biomarkers using an antibody specific for the combination of biomarkers in the sample of step a), and
c) comparing the presence and/or amount of said combination of biomarkers obtained from step b) to the presence and/or amount of said combination of biomarkers in a control sample, to identify an increased risk for preterm delivery before 32 weeks of gestation,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for diagnosing whether an individual is at risk of imminent birth, the method comprising: a) obtaining a sample of vaginal fluid from an individual,
b) determining the presence and/or amount of the combination of biomarkers using an antibody specific for the combination of biomarkers in the sample of step a), and
c) comparing the presence and/or amount of said combination of biomarkers obtained from step b) to the presence and/or amount of said combination of biomarkers in a control sample, to identify an increased risk of imminent birth,
wherein the combination of biomarkers is COL1A1, CCN5, CCL18, CCL2, IL1RL1, CD14, CD163 and TNFRSF8.

In another embodiment, the present invention relates to a method for diagnosing, whether an individual is at risk of preterm delivery, the method comprising:
a) obtaining a sample of vaginal fluid from an individual,
b) determining the presence and/or amount of the combination of biomarkers using an oligonucleotide specific for the combination of biomarkers in the sample of step a), and
c) comparing the presence and/or amount of said combination of biomarkers obtained from step b) to the presence and/or amount of said combination of biomarkers in a control sample, to identify an increased risk for preterm delivery,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for diagnosing, whether an individual is at risk of preterm delivery before 32 weeks of gestation, the method comprising:
a) obtaining a sample of vaginal fluid from an individual,
b) determining the presence and/or amount of the combination of biomarkers using an oligonucleotide specific for the combination of biomarkers in the sample of step a), and
c) comparing the presence and/or amount of said combination of biomarkers obtained from step b) to the presence and/or amount of said combination of biomarkers in a control sample, to identify an increased risk for preterm delivery before 32 weeks of gestation,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for diagnosing, whether an individual is at risk of imminent birth, the method comprising:
a) obtaining a sample of vaginal fluid from an individual,
b) determining the presence and/or amount of the combination of biomarkers using an oligonucleotide specific for the combination of biomarkers in the sample of step a), and
c) comparing the presence and/or amount of said combination of biomarkers obtained from step b) to the presence and/or amount of said combination of biomarkers in a control sample, to identify an increased risk of imminent birth,
wherein the combination of biomarkers is COL1A1, CCN5, CCL18, CCL2, IL1RL1, CD14, CD163 and TNFRSF8.

In one embodiment, the present invention relates to a method for prognosing whether an individual is at risk of preterm delivery, the method comprising:
a) determining the presence and/or amount of the combination of biomarkers using an antibody specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk for preterm delivery,
wherein the combination of biomarkers is COL1A1, CCN5, ALDH1A1, CCL18, CCL2, CCL13, IL1RL1, CD14, CD163 and TNFRSF8.

In one embodiment, the present invention relates to a method for prognosing whether an individual is at risk of preterm delivery before 32 weeks of gestation, the method comprising:
a) determining the presence and/or amount of the combination of biomarkers using an antibody specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk for preterm delivery before 32 weeks of gestation,
wherein the combination of biomarkers is COL1A1, CCN5, ALDH1A1, CCL18, CCL2, CCL13, IL1RL1, CD14, CD163 and TNFRSF8.

In one embodiment, the present invention relates to a method for prognosing whether an individual is at risk of imminent birth, the method comprising:
a) determining the presence and/or amount of the combination of biomarkers using an antibody specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk of imminent birth,
wherein the combination of biomarkers is COL1A1, CCN5, CCL18, CCL2, IL1RL1, CD14, CD163 and TNFRSF8.

In another embodiment, the present invention relates to a method for prognosing whether an individual is at risk of preterm delivery, the method comprising:
a) determining the presence and/or amount of the combination of biomarkers using an oligonucleotide specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk for preterm delivery,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for prognosing whether an individual is at risk of preterm delivery before 32 weeks of gestation, the method comprising:
a) determining the presence and/or amount of the combination of biomarkers using an oligonucleotide specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk for preterm delivery before 32 weeks of gestation,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for prognosing whether an individual is at risk of imminent birth, the method comprising:
a) determining the presence and/or amount of the combination of biomarkers using an oligonucleotide specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk of imminent birth,
wherein the combination of biomarkers is COL1A1, CCN5, CCL18, CCL2, IL1RL1, CD14, CD163 and TNFRSF8.

In another embodiment, the present invention relates to a method for diagnosing whether an individual is at risk of preterm delivery, the method comprising:
a) determining the presence and/or amount of the combination of biomarkers using an antibody specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk for preterm delivery,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for diagnosing whether an individual is at risk of preterm delivery before 32 weeks of gestation, the method comprising:
a) determining the presence and/or amount of the combination of biomarkers using an antibody specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk for preterm delivery before 32 weeks of gestation,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for diagnosing whether an individual is at risk of imminent birth, the method comprising:
a) determining the presence and/or amount of the combination of biomarkers using an antibody specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk of imminent birth, wherein the combination of biomarkers is COL1A1, CCN5, CCL18, CCL2, IL1RL1, CD14, CD163 and TNFRSF8.

In another embodiment, the present invention relates to a method for diagnosing, whether an individual is at risk of preterm delivery, the method comprising:
a) determining the presence and/or amount of the combination of biomarkers using an oligonucleotide specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk for preterm delivery, wherein the combination of biomarkers is COL1A1, CCN5, ALDH1A1, CCL18, CCL2, CCL13, IL1RL1, CD14, CD163 and TNFRSF8.

In another embodiment, the present invention relates to a method for diagnosing, whether an individual is at risk of preterm delivery before 32 weeks of gestation, the method comprising:
a) determining the presence and/or amount of the combination of biomarkers using an oligonucleotide specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk for preterm delivery before 32 weeks of gestation,
wherein the combination of biomarkers is COL1A1, CCN5, ALDH1A1, CCL18, CCL2, CCL13, IL1RL1, CD14, CD163 and TNFRSF8.

In another embodiment, the present invention relates to a method for diagnosing, whether an individual is at risk of imminent birth, the method comprising:
a) determining the presence and/or amount of the combination of biomarkers using an oligonucleotide specific for the combination of biomarkers in the sample of vaginal fluid from an individual, and
b) comparing the presence and/or amount of said combination of biomarkers obtained from step a) to the presence and/or amount of said combination of biomarkers in a control sample, to identify an increased risk of imminent birth,
wherein the combination of biomarkers is COL1A1, CCN5, CCL18, CCL2, IL1RL1, CD14, CD163 and TNFRSF8.

Advantageously, the preterm delivery is a preterm delivery before 32 weeks of gestation.

The invention also provides kits or devices that are useful in determining the risk of preterm delivery, advantageously useful in determining the risk of preterm delivery before 32 weeks of gestation or in determining the risk of imminent birth. The kits and devices of the present invention comprise at least the combination of biomarkers of the invention and/or one or more agent for the detection of or for the determination of the amount of the biomarkers of the combination of the invention. Specific biomarkers and agents for the detection of said biomarkers useful in the present invention are set forth herein.

Generally, the agents for the detection of the kit and the device will bind, with at least some specificity, to the biomarkers contained in the sample from which the combination of biomarkers is analyzed. Examples of classes of agents of the kit or device include, but are not to, proteins (including antibodies of the invention), and fragments thereof, peptides, polypeptides, proteoglycans, glycoproteins, lipoproteins, carbohydrates, lipids, nucleic acids, organic and inorganic chemicals, and natural and synthetic polymers. The agents for the detection of the biomarkers of the combination may be part of an array, or the agents may be packaged separately and/or individually. The agents may be immobilized on an inert support.

The kits and devices of the present invention also may contain reagents that can be used to detectably label biomarkers contained in the samples from which the combination of biomarkers is analyzed. For this purpose, the kit or device may comprise a set of antibodies or functional fragments thereof that specifically bind at least two, three, four, five, six, seven, eight, nine, up to all 10 of the biomarkers selected among the group comprising COL1A1, CCN5 ALDH1A1, CCL18, CCL2, CCL13, IL1RL1, CD14, CD163 and TNFRSF8. The antibodies themselves may be detectably labelled. The kit or device also may comprise a specific biomarker binding component, such as an aptamer.

In one embodiment, the present invention further provides a device for carrying out the use of the invention or for use in the method of the invention, which comprises:
a) one or more antibody and/or oligonucleotide specific for the combination of COL1A1 and CCN5 biomarkers for preterm delivery, and optionally,
b) one or more antibody and/or oligonucleotide specific for the combination of one or more additional biomarkers for imminent birth, wherein the one or more additional biomarkers is selected from the group comprising ALDH1A1, CCL18, CCL2, CCL13, IL1RL1, CD14, CD163 and TNFRSF8, and optionally
c) at least one internal standard.

In an advantageous embodiment, the kit or device of the invention comprises (a) one or more antibody specific for the combination of COL1A1 and CCN5 biomarkers for preterm delivery.

In another advantageous embodiment, the kit or device of the invention comprises (a) one or more oligonucleotide specific for the combination of COL1A1 and CCN5 biomarkers for preterm delivery.

In an advantageous embodiment, the kit or device of the invention comprises (a) one or more antibody specific for the combination of COL1A1 and CCN5 biomarkers for preterm delivery and (b) one or more antibody specific for the combination of one or more additional biomarkers for imminent birth, wherein the one or more additional biomarkers is selected from the group comprising ALDH1A1, CCL18, CCL2, CCL13, IL1RL1, CD14, CD163 and TNFRSF8.

In an advantageous embodiment, the kit or device of the invention comprises (a) one or more antibody specific for the combination of COL1A1 and CCN5 biomarkers for preterm delivery and (b) one or more antibody specific for the combination of one or more additional biomarkers for imminent birth, wherein the one or more additional biomarkers is selected from the group comprising ALDH1A1, CCL18, CCL2, CCL13, IL1RL1, CD14, CD163 and TNFRSF8, and (c) at least one internal standard.

In another advantageous embodiment, the kit or device of the invention comprises (a) one or more oligonucleotide specific for the combination of COL1A1 and CCN5 biomarkers for preterm delivery and (b) one or more oligonucleotide specific for the combination of one or more additional biomarkers for imminent birth, wherein the one or more additional biomarkers is selected from the group comprising ALDH1A1, CCL18, CCL2, CCL13, IL1RL1, CD14, CD163 and TNFRSF8.

In another advantageous embodiment, the kit or device of the invention comprises (a) one or more oligonucleotide specific for the combination of COL1A1 and CCN5 biomarkers for preterm delivery and (b) one or more oligonucleotide specific for the combination of one or more additional biomarkers for imminent birth, wherein the one or more additional biomarkers is selected from the group comprising ALDH1A1, CCL18, CCL2, CCL13, IL1RL1, CD14, CD163 and TNFRSF8 and (c) at least one internal standard.

As used herein, the at least one internal standard can be a negative control, such as stratifin (SFN).

In an advantageous embodiment, when used for determining the risk of preterm delivery, advantageously useful in determining the risk of preterm delivery before 32 weeks of gestation, the kit or device of the invention comprises (a) one or more antibody and/or oligonucleotide specific for the combination of COL1A1 and CCN5 biomarkers for preterm delivery and (b) one or more antibody and/or oligonucleotide specific for the combination of one or more additional biomarkers for imminent birth, wherein the one or more additional biomarkers is selected from the group comprising ALDH1A1, CCL18, CCL2, CCL13, IL1RL1, CD14, CD163 and TNFRSF8, and (c) at least one internal standard.

In an advantageous embodiment, when used for determining the risk of preterm delivery, advantageously useful in determining the risk of imminent birth, the kit or device of the invention comprises (a) one or more antibody and/or oligonucleotide specific for the combination of COL1A1 and CCN5 biomarkers for preterm delivery and (b) one or more antibody and/or oligonucleotide specific for the combination of one or more additional biomarkers for imminent birth, wherein the one or more additional biomarkers is selected from the group comprising CCL18, CCL2, IL1RL1, CD14, CD163 and TNFRSF8, and (c) at least one internal standard.

If the biomarkers comprise a nucleic acid, the kit or device may provide one or more oligonucleotide probe that is capable of forming a duplex with the biomarkers of the combination or with a complementary strand of said biomarkers of the combination. The one or more oligonucleotide probe may be detectably labelled. Typically, the one or more oligonucleotide probe used in the methods of the invention is selected from one or more of the oligonucleotide described herein.

The kits and devices of the present invention may also include pharmaceutical excipients, diluents and/or adjuvants when the biomarker is to be used to raise an antibody. Examples of pharmaceutical adjuvants include, but are not limited to, preservatives, wetting agents, emulsifying agents, and dispersing agents. The kit may additionally include standards or controls. The kit may additionally include buffers, diluents or other reagents, such as stop buffer, sample preparation buffer, colour development reagents, streptavidin conjugates, substrates or wash buffer.

The kit may comprise a device for obtaining or processing a vaginal fluid sample. The kit may comprise vaginal fluid extraction buffer, for example a buffer containing approximately 50 mM HEPES, 150 mM NaCl, 0.1% SDS, 1 mM EDTA, 1 mM Pefabloc SC 4-(2-aminoethyl_benzene sulfonyl fluoride (AEBSF). The kit may comprise a sample collection device, such as a swab, cervicovaginal wick, diaphragm-like device, cervical aspirator, or cytobrush. The kit may comprise a container suitable for storing a vaginal fluid sample.

Prevention of the action of microorganisms can be ensured by the inclusion of various antibacterial and antifungal agents, for example, paraben, chlorobutanol, phenol sorbic acid, and the like. It may also be desirable to include isotonic agents such as sugars, sodium chloride, and the like.

In one embodiment, the kits or devices are described above are particularly useful in determining the risk of preterm delivery before 32 weeks of gestation.

In one embodiment, the kits or devices are described above are particularly useful in determining the risk of imminent birth.

### EXAMPLES

### Example 1: Identification of COL1A1 and CCN5 as Specific Biomarkers for Preterm Delivery Before 32 Weeks of Gestation

1. Sample Received and Analyzed

105 vaginal samples, obtained from women with symptomatic preterm labor between 24 et 32^{6/7} weeks of gestation, were analyzed for identifying specific biomarkers for preterm delivery. Among the 105 samples, 9 samples have been obtained from women who subsequently delivered before 32 weeks of gestation (case group) and 96 who subsequently delivered after 32 weeks of gestation (control group).

2. Immunoassay Analysis

2.1. Col1A1 Biomarker:

The MSD assay was set up according to the following protocol:

Coating: A High-Bind MSD large spot plate was spotted with 30 μl per well of the capture (monoclonal) antibody at 2 μg/ml in PBS. The plate was incubated at 4° C. overnight without shaking. The plate was washed two times with 150 μl per well of PBS+0.05% Tween 20.

Blocking: A solution of 5% MSD blocker A in PBS was prepared for use as a blocking agent. 150 μl of blocking buffer was added to all wells of the coated plate and the plate was incubated for 45 minutes at room temperature with vigorous shaking. The plate was then washed two times with 150 μl of PBS+0.05% Tween 20.

Sample: Vaginal samples was determined in PBS-1% Blocker A and 25 μl/well was loaded. The plate was incubated for 2 hours at room temperature with shaking before being washed three times with 150 μl of PBS+0.05% Tween 20.

Detection: Polyclonal antibody anti-COL1A1 at 0.1 μg/ml in PBS+1% Blocker A was added at 25 μl/well. Plate was incubated for 2 hours at room temperature with shaking. Plate was then washed two times with 150 μl of PBS+0.05% Tween 20.

Streptavidin SulfoTAG: Streptavidin SulfoTAG was prepared at 0.5 μg/ml in PBS+1% Blocker A. 25 μl of Streptavidin SulfoTAG reagent was added to all wells and the plate was incubated for 45 minutes at room temperature with shaking. Plate was then washed two times with 150 μl of PBS+0.05% Tween 20.

Reading: 150 μl of MSD Reading Buffer at 2× concentration was added to all wells and the plate was read immediately on the SectorImager 6000

Results:

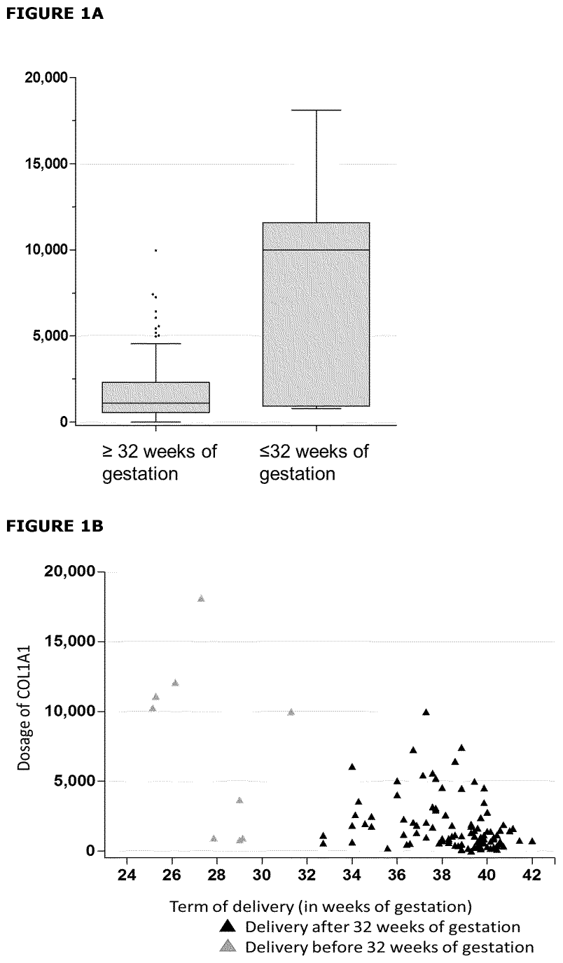

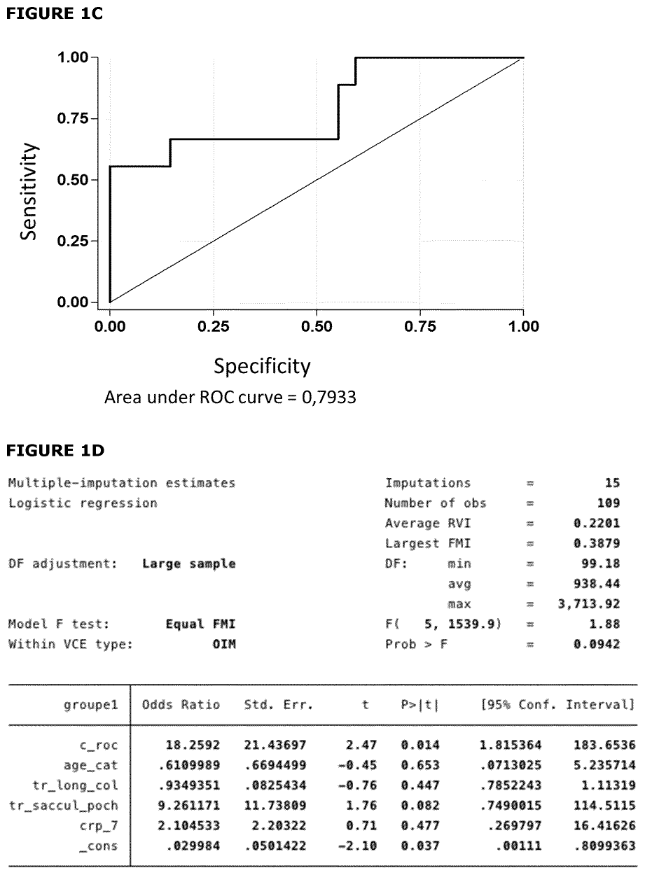

Results are presented in FIGS. 1A to 1D.

Conclusion:

COL1A1 protein level was increased in the group where the delivery birth was before 32 weeks gestation, wherein the median of the case group is 9993.8 μg/ml versus 1107.9 μg/ml for the control group (see FIG. 1A). It thus results that COL1A1 is a biomarker for preterm delivery, in particular COL1A1 is a biomarker for preterm delivery before 32 weeks of gestation.

2.2. CCN5 Biomarker

The MSD assay was set up according to the following protocol:

Coating: A High-Bind MSD large spot plate was spotted with 30 μl per well of the capture (monoclonal) antibody at 1 μg/ml in PBS. The plate was incubated at 4° C. overnight without shaking. The plate was washed two times with 150 μl per well of PBS+0.05% Tween 20.

Blocking: A solution of 5% MSD blocker A in PBS was prepared for use as a blocking agent. 150 μl of blocking buffer was added to all wells of the coated plate and the plate was incubated for 45 minutes at room temperature with vigorous shaking. The plate was then washed two times with 150 μl of PBS+0.05% Tween 20.

Sample: Vaginal samples was determined in PBS-1% Blocker A and 25 μl/well was loaded. The plate was incubated for 2 hours at room temperature with shaking before being washed three times with 150 μl of PBS+0.05% Tween 20.

Detection: Polyclonal antibody anti-CCN5 at 0.4 μg/ml in PBS+1% Blocker A was added at 25 μl/well. Plate was incubated for 2 hours at room temperature with shaking. Plate was then washed two times with 150 μl of PBS+0.05% Tween 20.

Streptavidin SulfoTAG: Streptavidin SulfoTAG was prepared at 0.5 μg/ml in PBS+1% Blocker A. 25 μl of Streptavidin SulfoTAG reagent was added to all wells and the plate was incubated for 45 minutes at room temperature with shaking. Plate was then washed two times with 150 μl of PBS+0.05% Tween 20.

Reading: 150 μl of MSD Reading Buffer at 2× concentration was added to all wells and the plate was read immediately on the SectorImager 6000

Results:

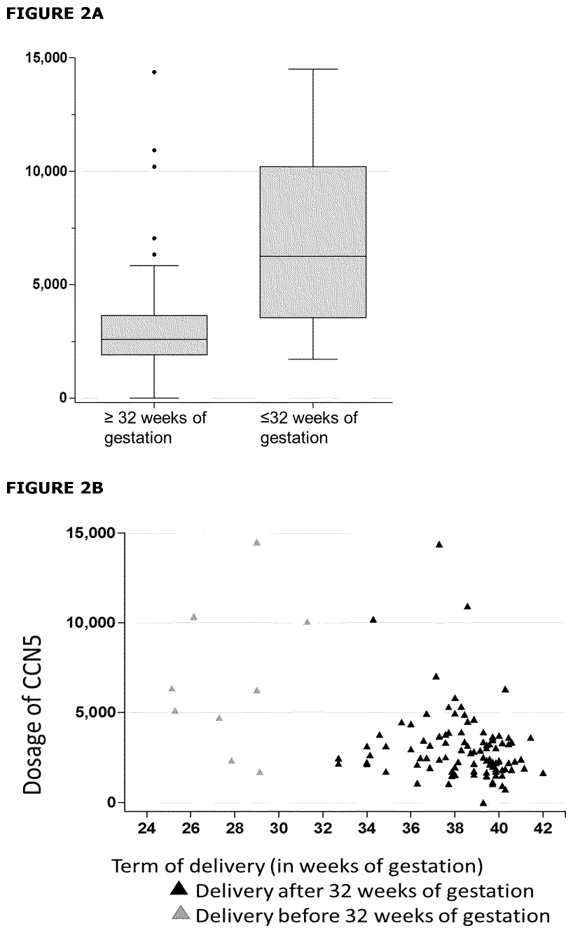

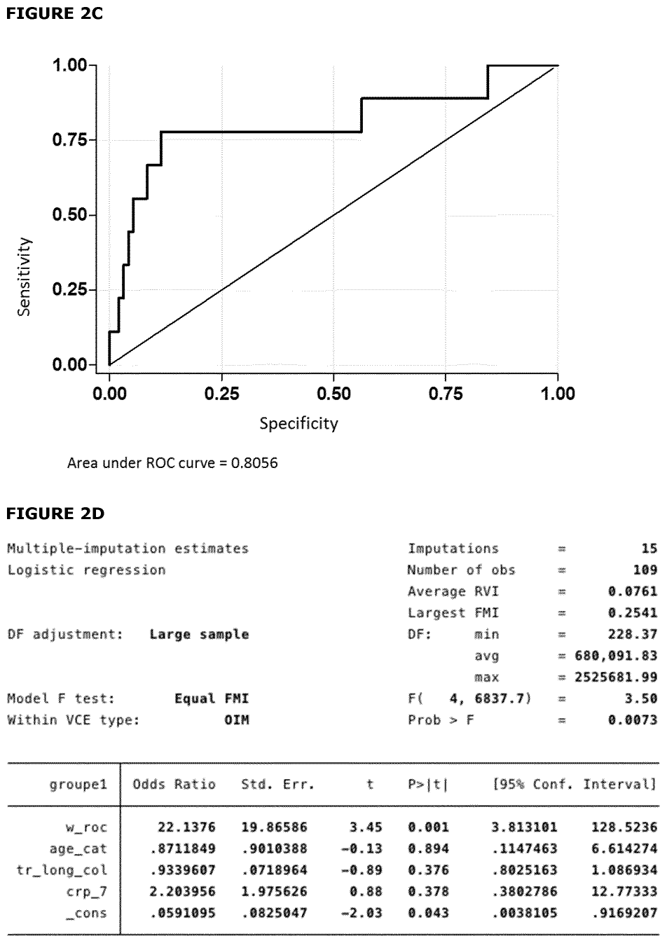

Results are presented in FIGS. 2A to 2D.

Conclusion:

CCN5 protein level was increased in the group where the delivery birth was before 32 weeks gestation, wherein the median of the case group is 6240 pg/ml versus 2514 pg/ml for the control group (see FIG. 2A). It thus results that CCN5 is a biomarker for preterm delivery, in particular a biomarker for preterm delivery before 32 weeks of gestation.

### Example 2: Identification of ALDH1A1, CCL18, CCL2, CCL13, IL1RL1, CD14, CD163 and TNFRSF8 as Specific Biomarkers for Imminent Spontaneous Delivery

1. Sample Received and Analyzed

58 vaginal samples were analyzed for identifying specific biomarkers for imminent spontaneous birth.

The repartition of the 58 samples is described in Table 1:

TABLE 1
Repartition of the 58 vaginal samples.

| | | | Premature |
|---|---|---|---|
| Group of vaginal | Spontaneous | Induced | rupture of |
| samples | delivery | delivery | membranes |
| Vaginal samples at | 12 | 8 | 9 |
| the third quarter of | | | |
| gestation | | | |
| Vaginal samples at | 15 | 7 | 7 |
| term | | | |

The presence and the concentration of each biomarker ALDH1A1, CCL18, CCL2, CCL13, IL1RL1, CD14, CD163 and TNFRSF8 were tested in all samples.

2. Immunoassay Analysis

The Luminex assay was set up according to the following protocol:

50 μL of standard or sample was added per well. A plate layout is provided to record standards and samples assayed. The diluted Microparticle Cocktail was resuspended by inversion or vortexing.

50 μL of the microparticle cocktail was added to each well of the microplate. The microplate was then incubated for 2 hours at room temperature on a horizontal orbital microplate shaker (0.12″ orbit) set at 800±50 rpm.

A magnetic device designed to accommodate a microplate was used. Wash was realized by applying the magnet to the bottom of the microplate for 1 minute. The liquid was then removed. Each well was filing with Wash Buffer (100 μL) and the liquid was removed 1 minute later. The wash procedure was repeated three times. 50 μL of diluted Biotin-Antibody Cocktail was added to each well.

The plate was then incubated for 1 hour at room temperature on the shaker set at 800±50 rpm. A magnetic device designed to accommodate a microplate was used. Wash was realized by applying the magnet to the bottom of the microplate for 1 minute. The liquid was then removed.

Each well was filing with Wash Buffer (100 μL) and the liquid was removed 1 minute later. The wash procedure was repeated three times. 50 μL of diluted Streptavidin-PE was added to each well. The plate was then incubated for 30 minutes at room temperature on the shaker set at 800±50 rpm.

A magnetic device designed to accommodate a microplate was used. Wash was realized by applying the magnet to the bottom of the microplate for 1 minute. The liquid was then removed. Each well was filing with Wash Buffer (100 μL) and the liquid was removed 1 minute later. The wash procedure was repeated three times. Microparticles were resuspended by adding 100 μL of Wash Buffer to each well and incubate for 2 minutes on the shaker set at 800±50 rpm.

The reading was realized within 90 minutes using a Luminex® or Bio-Rad analyzer.

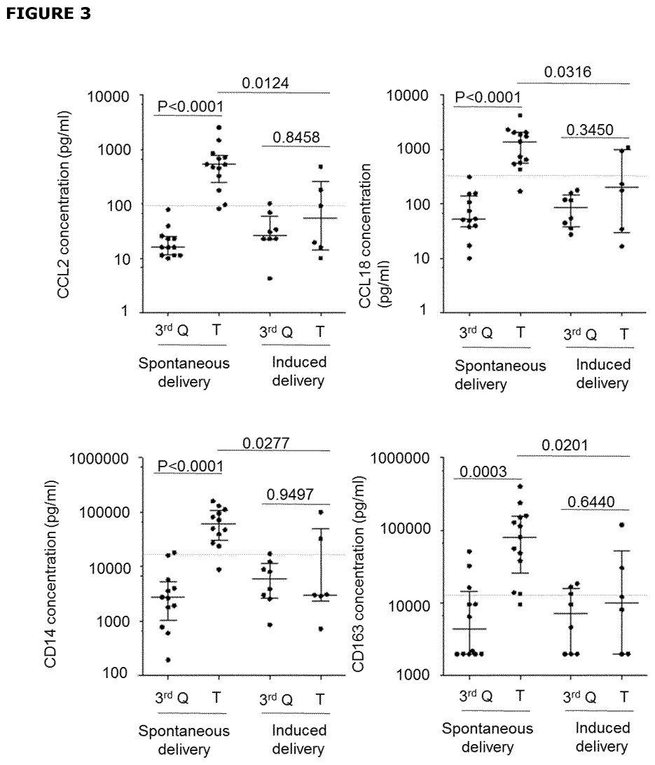

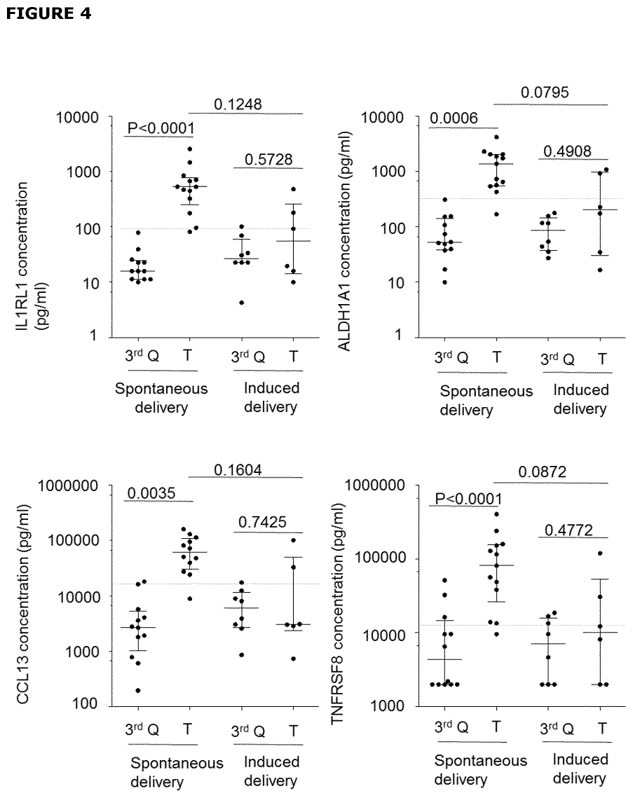

Results: Results are presented in FIGS. 3 and 4.

Table 2 summarizes the critic value obtained for each of the tested biomarkers

TABLE 2
Critical value obtained for each of the tested biomarkers

| Tested | | | | | | | | |
|---|---|---|---|---|---|---|---|---|
| biomarkers | ALDH1A1 | CCL18 | CCL2 | CCL13 | IL1RL1 | CD14 | CD163 | TNFRSF8 |
| Critical | 1171 | 327.3 | 93.41 | 3.615 | 9319 | 16514 | 12791 | 3.355 |
| value | | | | | | | | |
| (pg/ml) | | | | | | | | |

Conclusion:

The biomarkers ALDH1A1, CCL18, CCL2, CCL13, IL1RL1, CD14, CD163 and TNFRSF8 are biomarkers for imminent spontaneous childbirth.

### Example 3: Identification of Stratifin (SFN) as Internal Standard

1. Sample Received and Analyzed

105 vaginal samples, obtained from women with symptomatic preterm labor between 24 et 32^{6/7} weeks of gestation, were analyzed for identifying specific biomarkers for preterm delivery. Among the 105 samples, 9 samples have been obtained from women who subsequently delivered before 32 weeks of gestation (case group) and 96 who subsequently delivered after 32 weeks of gestation (control group).

2. Immunoassay Analysis: SFN as Internal Standard

The MSD assay was set up according to the following protocol:

Coating: A High-Bind MSD large spot plate was spotted with 30 μl per well of the capture (monoclonal) antibody at 2 μg/ml in PBS. The plate was incubated at 4° C. overnight without shaking. The plate was washed two times with 150 μl per well of PBS+0.05% Tween 20.

Blocking: A solution of 5% MSD blocker A in PBS was prepared for use as a blocking agent. 150 μl of blocking buffer was added to all wells of the coated plate and the plate was incubated for 45 minutes at room temperature with vigorous shaking. The plate was then washed two times with 150 μl of PBS+0.05% Tween 20.

Sample: Vaginal samples was determined in PBS-1% Blocker A and 25 μl/well was loaded. The plate was incubated for 2 hours at room temperature with shaking before being washed three times with 150 μl of PBS+0.05% Tween 20.

Detection: Polyclonal antibody anti-SFN at 0.1 μg/ml in PBS+1% Blocker A was added at 25 μl/well. Plate was incubated for 2 hours at room temperature with shaking. Plate was then washed two times with 150 μl of PBS+0.05% Tween 20.

Streptavidin SulfoTAG: Streptavidin SulfoTAG was prepared at 0.5 μg/ml in PBS+1% Blocker A. 25 μl of Streptavidin SulfoTAG reagent was added to all wells and the plate was incubated for 45 minutes at room temperature with shaking. Plate was then washed two times with 150 μl of PBS+0.05% Tween 20.

Reading: 150 μl of MSD Reading Buffer at 2× concentration was added to all wells and the plate was read immediately on the SectorImager 6000

Results:

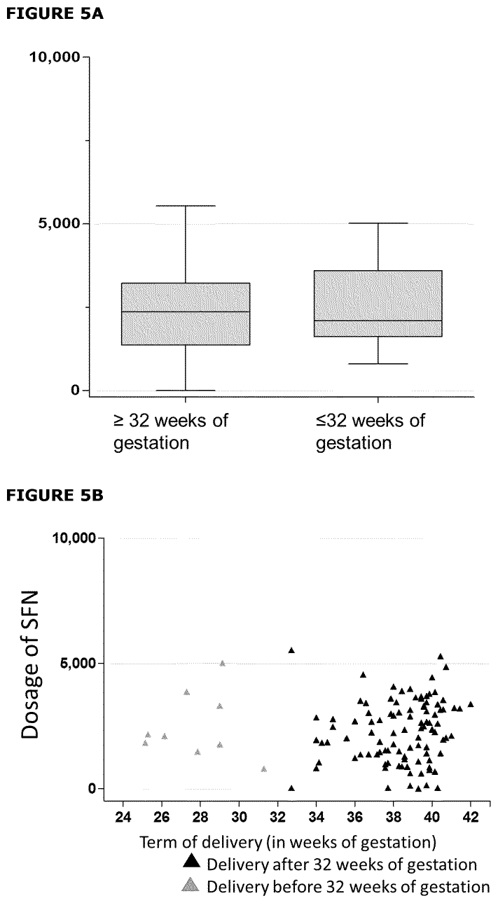

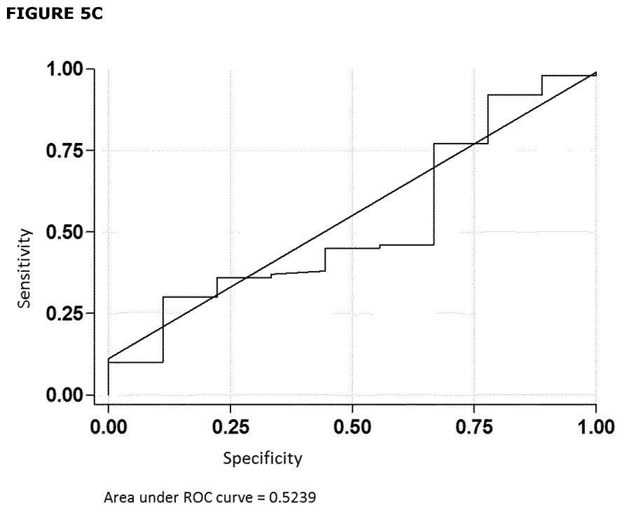

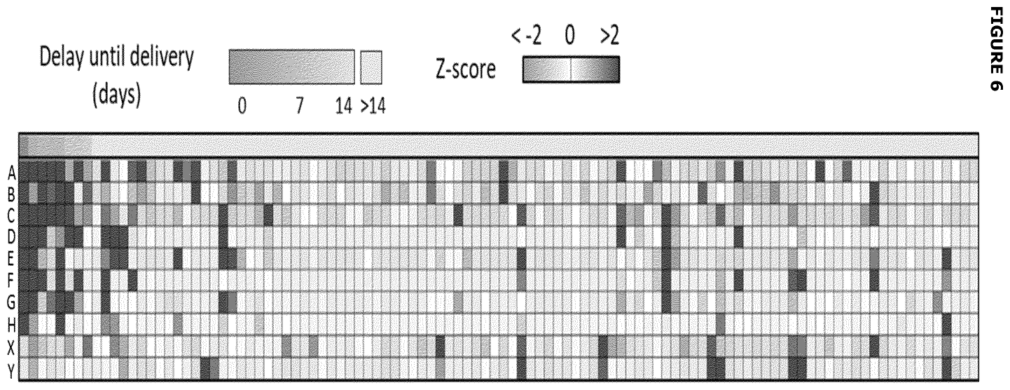

Results are presented in FIGS. 5A to 5C.

Conclusion:

SFN protein level was equivalent between the group where the delivery birth was before 32 weeks gestation and the control group (see FIG. 5A). It thus results that SFN can be used as internal standard.

### Example 4: Identification of COL1A1, CCN5, ALDH1A1, CCL2, CCL13, IL1R1, CD14, TNFRSF8, CCL18, and CD163 as Specific Biomarkers for Preterm Delivery within 7 Days

105 vaginal samples, obtained from women with symptomatic preterm labor between 24 et 32^{6/7} weeks of gestation, were analyzed for identifying specific biomarkers for preterm delivery within 7 days after consulting for PTL. Among the 105 samples, 2 samples have been obtained from women who subsequently delivered within 7 days (case group) and 103 who subsequently delivered after 7 days (control group).

1. Col1A1 Biomarker:

The MSD assay was set up according to the following protocol:

Coating: A High-Bind MSD large spot plate was spotted with 30 μl per well of the capture (monoclonal) antibody at 2 μg/ml in PBS. The plate was incubated at 4° C. overnight without shaking. The plate was washed two times with 150 μl per well of PBS+0.05% Tween 20.

Blocking: A solution of 5% MSD blocker A in PBS was prepared for use as a blocking agent. 150 μl of blocking buffer was added to all wells of the coated plate and the plate was incubated for 45 minutes at room temperature with vigorous shaking. The plate was then washed two times with 150 μl of PBS+Tween 20.

Sample: Vaginal samples was determined in PBS-1% Blocker A and 25 μl/well was loaded. The plate was incubated for 2 hours at room temperature with shaking before being washed three times with 150 μl of PBS+0.05% Tween 20.

Detection: Polyclonal antibody anti-COL1A1 at 0.1 μg/ml in PBS+1% Blocker A was added at 25 μl/well. Plate was incubated for 2 hours at room temperature with shaking. Plate was then washed two times with 150 μl of PBS+0.05% Tween 20.

Streptavidin SulfoTAG: Streptavidin SulfoTAG was prepared at 0.5 μg/ml in PBS+1% Blocker A. 25 μl of Streptavidin SulfoTAG reagent was added to all wells and the plate was incubated for 45 minutes at room temperature with shaking. Plate was then washed two times with 150 μl of PBS+0.05% Tween 20.

Reading: 150 μl of MSD Reading Buffer at 2× concentration was added to all wells and the plate was read immediately on the SectorImager 6000.

2. CCN5 Biomarker

The MSD assay was set up according to the following protocol:

Coating: A High-Bind MSD large spot plate was spotted with 30 μl per well of the capture (monoclonal) antibody at 1 μg/ml in PBS. The plate was incubated at 4° C. overnight without shaking. The plate was washed two times with 150 μl per well of PBS+0.05% Tween 20.

Blocking: A solution of 5% MSD blocker A in PBS was prepared for use as a blocking agent. 150 μl of blocking buffer was added to all wells of the coated plate and the plate was incubated for 45 minutes at room temperature with vigorous shaking. The plate was then washed two times with 150 μl of PBS+Tween 20.

Sample: Vaginal samples was determined in PBS-1% Blocker A and 25 μl/well was loaded. The plate was incubated for 2 hours at room temperature with shaking before being washed three times with 150 μl of PBS+0.05% Tween 20.

Detection: Polyclonal antibody anti-CCN5 at 0.4 μg/ml in PBS+1% Blocker A was added at 25 μl/well. Plate was incubated for 2 hours at room temperature with shaking. Plate was then washed two times with 150 μl of PBS+0.05% Tween 20.

Streptavidin SulfoTAG: Streptavidin SulfoTAG was prepared at 0.5 μg/ml in PBS+1% Blocker A. 25 μl of Streptavidin SulfoTAG reagent was added to all wells and the plate was incubated for 45 minutes at room temperature with shaking. Plate was then washed two times with 150 μl of PBS+0.05% Tween 20.

Reading: 150 μl of MSD Reading Buffer at 2× concentration was added to all wells and the plate was read immediately on the SectorImager 6000.

3. ALDH1A1, CCl13, CCL18, CCL2, IL1RL1, CD14, CD163 and TNFRSF8 Biomarkers

It is reminder that:
MCP1 is also named CCL2,
IL33R is also named IL1RL1, and
CD30 is also named TNFRSF8.

The Luminex assay was set up according to the following protocol: 50 μL of standard or sample was added per well. A plate layout is provided to record standards and samples assayed. The diluted Microparticle Cocktail was resuspended by inversion or vortexing.

50 μL of the microparticle cocktail was added to each well of the microplate. The microplate was then incubated for 2 hours at room temperature on a horizontal orbital microplate shaker (0.12″ orbit) set at 800±50 rpm.

A magnetic device designed to accommodate a microplate was used. Wash was realized by applying the magnet to the bottom of the microplate for 1 minute.

The liquid was then removed. Each well was filing with Wash Buffer (100 μL) and the liquid was removed 1 minute later. The wash procedure was repeated three times. 50 μL of diluted Biotin-Antibody Cocktail was added to each well. The plate was then incubated for 1 hour at room temperature on the shaker set at 800±50 rpm. A magnetic device designed to accommodate a microplate was used. Wash was realized by applying the magnet to the bottom of the microplate for 1 minute. The liquid was then removed.

Each well was filing with Wash Buffer (100 μL) and the liquid was removed 1 minute later. The wash procedure was repeated three times. 50 μL of diluted Streptavidin-PE was added to each well. The plate was then incubated for 30 minutes at room temperature on the shaker set at 800±50 rpm.

A magnetic device designed to accommodate a microplate was used. Wash was realized by applying the magnet to the bottom of the microplate for 1 minute. The liquid was then removed. Each well was filing with Wash Buffer (100 μL) and the liquid was removed 1 minute later. The wash procedure was repeated three times. Microparticles were resuspended by adding 100 μL of Wash Buffer to each well and incubate for 2 minutes on the shaker set at 800±50 rpm.

The reading was realized within 90 minutes using a Luminex® or Bio-Rad analyzer.

Dosage of fetal fibronectin (fFN) and analyzed cervical length (CL) measurements performed at inclusion, when available.

Dosage of fetal fibronectin (fFN) was measured using the Human Fetal Fibronectin (fFN) ELISA of Cusabio®. The assay procedure was implemented according to the instructions notice.

Reagents preparation: Biotin-antibody, HRP-avidin, and washing buffer were prepared according to the instructions notice of Human Fetal Fibronectin (fFN) ELISA of Cusabio®.

Sample: Vaginal samples were collected and centrifuged for 15 minutes at 1000 g at a temperature comprised between 2 and 8° C. within 30 minutes. Then, the samples were assayed.

Briefly, reagent, samples and standards were prepared as instructed. 100 μl of sample was added in each well and incubated for 2 hours at 37° C. The liquid was then removed from each well. 100 μl of biotin-antibody was added in each well and incubated for 1 hour at 37° C. Each well was aspired and washed 3 times using a washing buffer. 100 μl of HRP-avidin was added in each well and incubated for 1 hour at 37° C. Each well was aspired and washed 5 times using a washing buffer. 90 μl of TMB Substrate was added in each well and incubated for 15 to 30 minutes at 37° C. 50 μl of Stop solution was added in each well and the results were read at 450 nm within 5 minutes.

Results are presented in tables 3 to 5

TABLE 3
Predictive values for preterm delivery before 32 weeks of gestation (n = 10, >33 weeks, n = 96)

| | ROC | | 95% | Threshold | | 95% | | 95% |
|---|---|---|---|---|---|---|---|---|
| Biomarkers | AUC | p | CI | Value | Sensitivity | CI | Specificity | CI |
| COL1A1 | 0.793 | 0.0003 | [0.70-0.86] | >3657 | 66.7 | [35.4-87.9] | 86 | [78.0-91.0] |
| CCN5 | 0.806 | 0.0005 | [0.72-0.88] | >4714 | 77.8 | [45.3-93.6] | 89 | [81.4-93.8] |
| STFN | 0.524 | 0.9223 | [0.33-0.71] | | | | | |
| MCP1 | 0.921 | <0.0001 | [0.82-1.02] | >46.00 | 87.5 | [47.3-99.7] | 89.58 | [81.7-94.9] |
| IL33R | 0.856 | 0.0009 | [0.70-1.01] | >390.5 | 87.5 | [47.3-99.7] | 78.13 | [68.5-85.9] |
| CD14 | 0.837 | 0.0016 | [0.70-0.97] | >1182 | 87.5 | [47.3-99.7] | 63.54 | [53.1-73.1] |
| CD30 | 0.843 | 0.0013 | [0.65-1.03] | >0.5000 | 75 | [34.9-96.8] | 86.46 | [77.9-92.6] |
| CCL18 | 0.824 | 0.0024 | [0.61-1.03] | >40.50 | 87.5 | [47.3-99.7] | 66.67 | [56.3-75.9] |
| CD163 | 0.839 | 0.0015 | [0.68-1.00] | >8877 | 87.5 | [47.3-99.7] | 75 | [65.1-83.3] |
| ALDH1A1 | 0.535 | 0.7452 | [0.27-0.89] | | | | | |
| CCL13 | 0.667 | 0.4386 | [0.31-1.03] | | | | | |

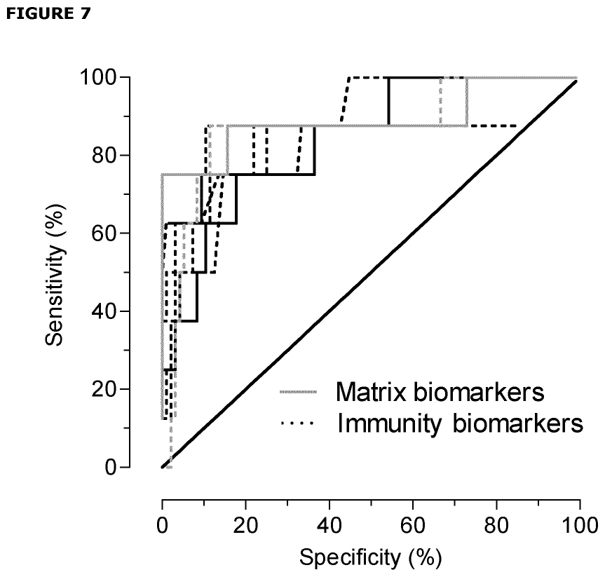

A z-score was calculated. Heatmap of the calculated z-scores showed a clear increase of biomarkers concentration in cervico-vaginal fluids of subjects with preterm delivery before 32 weeks of gestation (see FIGS. 6 and 7).

TABLE 4
Predictive values for preterm delivery within 7 days (Delivery within 7 days, n = 2; beyond 7 days, n = 103)

| | ROC | | 95% | Threshold | | 95% | | 95% |
|---|---|---|---|---|---|---|---|---|
| Biomarkers | AUC | P | CI | Value | Sensitivity | CI | Specificity | CI |
| COL1A1 | 0.986 | 0.0117 | [0.96-1.0] | >10669 | 100 | [15.8-100.0] | 98.08 | [93.2-99.8] |
| CCN5 | 0.933 | 0.0367 | [0.85-1.01] | >5036 | 100 | [15.8-100.0] | 88.46 | [80.7-93.9] |
| STFN | 0.543 | 0.8345 | [0.45-0.64] | | | | | |
| MCP1 | 0.995 | 0.0168 | [0.98-1.0] | >192.5 | 100 | [15.8-100.0] | 98.08 | [93.2-99.8] |
| IL33R | 0.990 | 0.0179 | [0.97-1.01] | >8393 | 100 | [15.8-100.0] | 98.08 | [93.2-99.8] |
| CD14 | 0.976 | 0.0216 | [0.94-1.01] | >14469 | 100 | [15.8-100.0] | 96.15 | [90.4-98.9] |
| CD30 | 1.000 | 0.0158 | [1.00-1.00] | >42.50 | 100 | [15.8-100.0] | 100 | [96.5-100.0] |
| CCL18 | 0.986 | 0.0191 | [0.96-1.02] | >417.0 | 100 | [15.8-100.0] | 97.12 | [91.8-99.4] |
| CD163 | 0.962 | 0.0259 | [0.92-1.01] | >85758 | 100 | [15.8-100.0] | 94.23 | [87.8-97.8] |
| ALDH1A1 | 0.549 | 0.8147 | [0.06-1.16] | | | | | |
| CCL13 | 0.732 | 0.2679 | [0.27-1.19] | | | | | |

TABLE 5
Predictive values for preterm delivery within 14 days (Delivery within 14 days, n = 10; beyond 14 days, n = 95)

| | ROC | | 95% | Threshold | | 95% | | 95% |
|---|---|---|---|---|---|---|---|---|
| Biomarkers | AUC | p | CI | Value | Sensitivity | CI | Specificity | CI |
| COL1A1 | 0.946 | <0.0001 | [0.89-0.99] | >3515 | 90 | [55.5-99.7] | 85.42 | [76.7-91.8] |
| CCN5 | 0.818 | 0.0010 | [0.64-0.99] | >4680 | 80 | [44.4-97.5] | 89.58 | [81.7-94.9] |
| STFN | 0.529 | 0.7622 | [0.38-0.67] | | | | | |
| MCP1 | 0.933 | <0.0001 | [0.87-0.99] | >42.50 | 90 | [55.5-99.7] | 87.5 | [79.2-93.4] |
| IL33R | 0.927 | <0.0001 | [0.86-0.99] | >390.5 | 90 | [55.5-99.7] | 79.17 | [69.7-86.8] |
| CD14 | 0.788 | 0.0028 | [0.65-0.92] | >1182 | 80 | [44.4-97.5] | 63.54 | [53.1-73.1] |
| CD30 | 0.869 | 0.0001 | [0.71-1.02] | >0.5000 | 80 | [44.4-97.5] | 88.54 | [80.4-94.1] |
| CCL18 | 0.879 | <0.0001 | [0.74-1.01] | >110.5 | 80 | [44.4-97.5] | 87.5 | [79.2-93.4] |
| CD163 | 0.832 | 0.0006 | [0.69-0.97] | >8877 | 80 | [44.4-97.5] | 75.0 | [65.1-83.3] |
| ALDH1A1 | 0.500 | 1.0000 | [0.18-0.82] | | | | | |
| CCL13 | 0.651 | 0.1503 | [0.43-0.87] | | | | | |

It thus results that the specific combination of biomarkers COL1A1, CCN5, ALDH1A1, CCL18, CCL2, CCL13 IL1RL1, CD14, CD163 and TNFRSF8 are useful for prognosing and/or to diagnosing, whether an individual is at risk of from preterm delivery.

### Example 5: Identification of COL1A1, CCN5, CCL2, IL1R1, CD14, TNFRSF8, CCL18, and CD163 as Specific Biomarkers for Imminent Birth within 7 Days

127 vaginal samples, obtained from women with symptomatic preterm delivery between 24 et 32^{6/7} weeks of gestation, were analyzed for identifying specific biomarkers for preterm delivery within 7 days after consulting for preterm delivery. Among the 127 samples, 30 samples have been obtained from women who subsequently delivered within 7 days (case group) and 97 who subsequently delivered after 7 days (control group).

1. Col1A1 Biomarker:

The MSD assay was set up according to the following protocol:

Coating: A High-Bind MSD large spot plate was spotted with 30 μl per well of the capture (monoclonal) antibody at 2 μg/ml in PBS. The plate was incubated at 4° C. overnight without shaking. The plate was washed two times with 150 μl per well of PBS+0.05% Tween 20.

Blocking: A solution of 5% MSD blocker A in PBS was prepared for use as a blocking agent. 150 μl of blocking buffer was added to all wells of the coated plate and the plate was incubated for 45 minutes at room temperature with vigorous shaking. The plate was then washed two times with 150 μl of PBS+Tween 20.

Sample: Vaginal samples was determined in PBS-1% Blocker A and 25 μl/well was loaded. The plate was incubated for 2 hours at room temperature with shaking before being washed three times with 150 μl of PBS+0.05% Tween 20.

Detection: Polyclonal antibody anti-COL1A1 at 0.1 μg/ml in PBS+1% Blocker A was added at 25 μl/well. Plate was incubated for 2 hours at room temperature with shaking. Plate was then washed two times with 150 μl of PBS+0.05% Tween 20.

Streptavidin SulfoTAG: Streptavidin SulfoTAG was prepared at 0.5 μg/ml in PBS+1% Blocker A. 25 μl of Streptavidin SulfoTAG reagent was added to all wells and the plate was incubated for 45 minutes at room temperature with shaking. Plate was then washed two times with 150 μl of PBS+0.05% Tween 20.

Reading: 150 μl of MSD Reading Buffer at 2× concentration was added to all wells and the plate was read immediately on the SectorImager 6000

2. CCN5 Biomarker

The MSD assay was set up according to the following protocol:

Coating: A High-Bind MSD large spot plate was spotted with 30 μl per well of the capture (monoclonal) antibody at 1 μg/ml in PBS. The plate was incubated at 4° C. overnight without shaking. The plate was washed two times with 150 μl per well of PBS+0.05% Tween 20.

Blocking: A solution of 5% MSD blocker A in PBS was prepared for use as a blocking agent. 150 μl of blocking buffer was added to all wells of the coated plate and the plate was incubated for 45 minutes at room temperature with vigorous shaking. The plate was then washed two times with 150 μl of PBS+Tween 20.

Sample: Vaginal samples was determined in PBS-1% Blocker A and 25 μl/well was loaded. The plate was incubated for 2 hours at room temperature with shaking before being washed three times with 150 μl of PBS+0.05% Tween 20.

Detection: Polyclonal antibody anti-CCN5 at 0.4 μg/ml in PBS+1% Blocker A was added at 25 μl/well. Plate was incubated for 2 hours at room temperature with shaking. Plate was then washed two times with 150 μl of PBS+0.05% Tween 20.

Streptavidin SulfoTAG: Streptavidin SulfoTAG was prepared at 0.5 μg/ml in PBS+1% Blocker A. 25 μl of Streptavidin SulfoTAG reagent was added to all wells and the plate was incubated for 45 minutes at room temperature with shaking. Plate was then washed two times with 150 μl of PBS+0.05% Tween 20.

Reading: 150 μl of MSD Reading Buffer at 2× concentration was added to all wells and the plate was read immediately on the SectorImager 6000.

3. CCL18, CCL2, IL1RL1, CD14, CD163 and TNFRSF8 Biomarkers

It is reminder that:
MCP1 is also named CCL2,
IL33R is also named IL1RL1, and
CD30 is also named TNFRSF8.

The Luminex assay was set up according to the following protocol:

50 μL of standard or sample was added per well. A plate layout is provided to record standards and samples assayed. The diluted Microparticle Cocktail was resuspended by inversion or vortexing.

50 μL of the microparticle cocktail was added to each well of the microplate. The microplate was then incubated for 2 hours at room temperature on a horizontal orbital microplate shaker (0.12″ orbit) set at 800±50 rpm.

A magnetic device designed to accommodate a microplate was used. Wash was realized by applying the magnet to the bottom of the microplate for 1 minute. The liquid was then removed. Each well was filing with Wash Buffer (100 μL) and the liquid was removed 1 minute later. The wash procedure was repeated three times. 50 μL of diluted Biotin-Antibody Cocktail was added to each well.

The plate was then incubated for 1 hour at room temperature on the shaker set at 800±50 rpm. A magnetic device designed to accommodate a microplate was used. Wash was realized by applying the magnet to the bottom of the microplate for 1 minute. The liquid was then removed.

Each well was filing with Wash Buffer (100 μL) and the liquid was removed 1 minute later. The wash procedure was repeated three times. 50 μL of diluted Streptavidin-PE was added to each well. The plate was then incubated for 30 minutes at room temperature on the shaker set at 800±50 rpm.

A magnetic device designed to accommodate a microplate was used. Wash was realized by applying the magnet to the bottom of the microplate for 1 minute. The liquid was then removed. Each well was filing with Wash Buffer (100 μL) and the liquid was removed 1 minute later. The wash procedure was repeated three times. Microparticles were resuspended by adding 100 μL of Wash Buffer to each well and incubate for 2 minutes on the shaker set at 800±50 rpm.

The reading was realized within 90 minutes using a Luminex® or Bio-Rad analyzer.

Dosage of fetal fibronectin (fFN) and analyzed cervical length (CL) measurements performed at inclusion, when available. Fetal fibronectin and cervical length cut-off points were chosen in accordance with what is commonly reported in Schmitz et al., Selective use of fetal fibronectin detection after cervical length measurement to predict spontaneous preterm delivery in women with preterm labor, American Journal of Obstetrics and Gynecology, 2006, vol 194, pages 138-143.

4. Dosage of Fibronectin

Dosage of fetal fibronectin (fFN) was measured using the Human Fetal Fibronectin (fFN) ELISA of Cusabio®. The assay procedure was implemented according to the instructions notice.

Reagents preparation: Biotin-antibody, HRP-avidin, and washing buffer were prepared according to the instructions notice of Human Fetal Fibronectin (fFN) ELISA of Cusabio®.

Sample: Vaginal samples were collected and centrifuged for 15 minutes at 1000 g at a temperature comprised between 2 and 8° C. within 30 minutes. Then, the samples were assayed.

Briefly, reagent, samples and standards were prepared as instructed. 100 μl of sample was added in each well and incubated for 2 hours at 37° C. The liquid was then removed from each well. 100 μl of biotin-antibody was added in each well and incubated for 1 hour at 37° C. Each well was aspired and washed 3 times using a washing buffer. 100 μl of HRP-avidin was added in each well and incubated for 1 hour at 37° C. Each well was aspired and washed 5 times using a washing buffer. 90 μl of TMB Substrate was added in each well and incubated for 15 to 30 minutes at 37° C. 50 μl of Stop solution was added in each well and the results were read at 450 nm within 5 minutes.

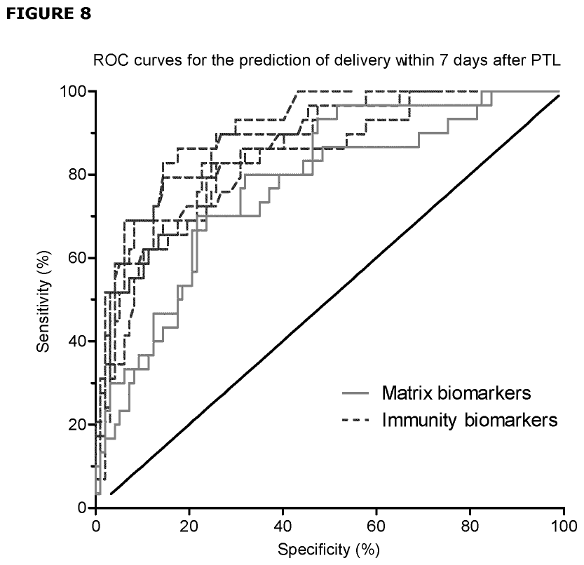

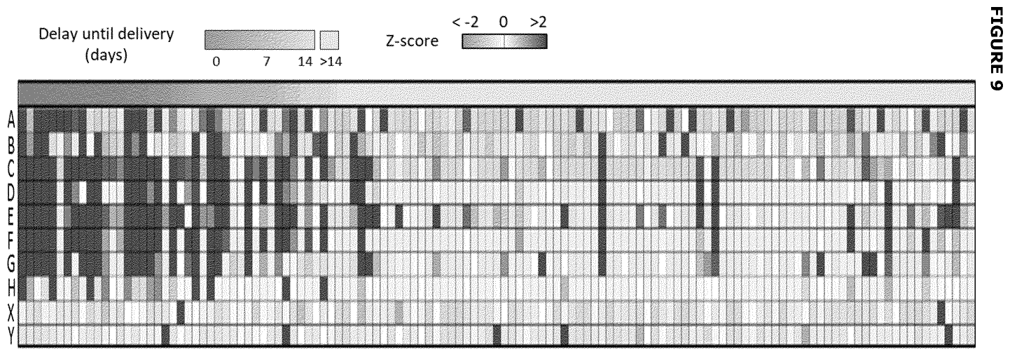

Results are presented in table 6 and FIG. 8.

TABLE 6
Predictive values for imminent birth within 7 days after preterm labor diagnosis

| | ROC | 95% | Sensitivity | 95% | Specificity | 95% | Cut-off |
|---|---|---|---|---|---|---|---|
| Biomarkers | AUC | CI | % | CI | % | CI | values |
| COL1A1 | 0.753 | <0.0001 | [0.65-0.85] | >899.7 | 80 | [61.4-92.3] | 60.8 |
| CCN5 | 0.790 | <0.0001 | [0.70-0.87] | >92.79 | 80 | [61.4-92.3] | 68.1 |
| MCP1 | 0.907 | <0.0001 | [0.85-0.96] | >31.91 | 90 | [72.6-97.8] | 73 |
| IL33R | 0.864 | <0.0001 | [0.79-0.93] | >188.9 | 90 | [72.6-97.8] | 60 |
| CD14 | 0.862 | <0.0001 | [0.79-0.93] | >6889 | 90 | [72.6-97.8] | 74 |
| CD30 | 0.847 | <0.0001 | [0.75-0.94] | >0.0600 | 83 | [64.2-94.1] | 69 |
| CCL18 | 0.845 | <0.0001 | [0.76-0.93] | >14.47 | 90 | [72.6-97.8] | 46 |
| CD163 | 0.829 | <0.0001 | [0.74-0.92] | >430.1 | 90 | [72.6-97.8] | 61 |
| fFN | 0.699 | 0.001 | [0.58-0.81] | <50 | 80 | [61.4-92.3] | 37.5 |
| | | | | ng/mL | | | |
| CL | 0.752 | 0.001 | [0.62-0.88] | <15 | 73.7 | [48.8-90.8] | 60.5 |
| | | | | mm | | | |

A z-score was calculated. Heatmap of the calculated z-scores showed a clear increase of imminent biomarkers concentration in cervico-vaginal fluids of subjects who delivered within 7 days (see FIG. 9). Regardless of gestational age, 22/30 (73.3%) cervico-vaginal fluids show at least 3 biomarkers with a z-score >1, compared to 18/97 (18.5%) cervico-vaginal fluids of subjects who delivered more than 7 days after inclusion.

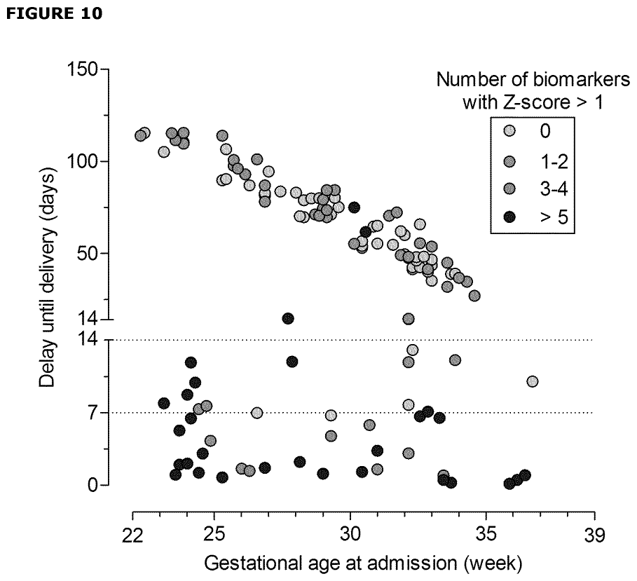

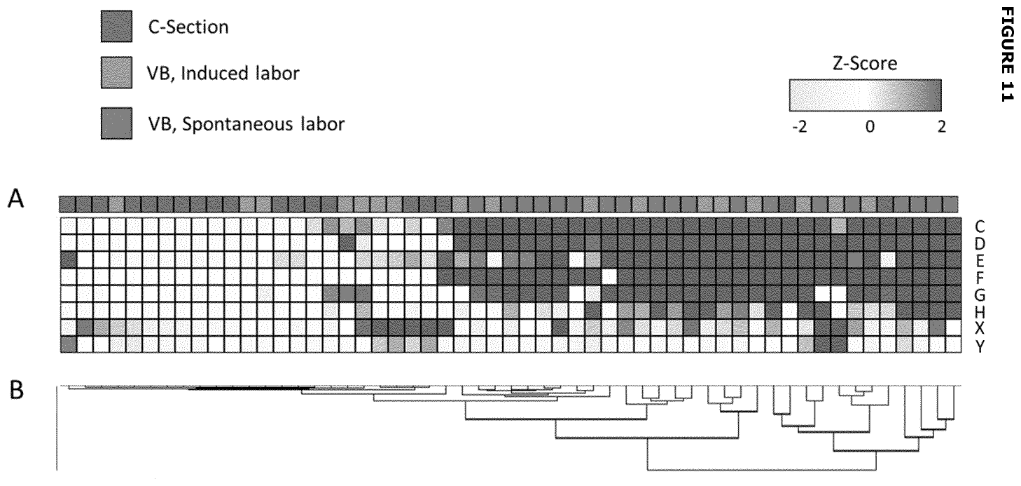

According to FIG. 10, in the cohort, 25/30 (83.3%) cervico-vaginal fluids contain at least 2 biomarkers with z-scores >1, compared to 23/97 (23.7%) cervico-vaginal fluids of subjects who delivered more than 7 days after admission.

It thus results that the specific combination of biomarkers COL1A1, CCN5, CCL18, CCL2, IL1RL1, CD14, CD163 and TNFRSF8 are useful for prognosing and/or to diagnosing, whether an individual is at risk of from imminent birth.

### Example 6: Distribution of Immunity Biomarkers and Subjects According to the Mode of Delivery at Term

The 6 immunity analytes CCL2 (C), IL1RL1 (D), CD14 (E), TNFRSF8 (F), CCL18 (G) and CD163 (H) and two negative controls TGFa (X) and CD66a (Y) were assayed in cervico-vaginal fluids from low-risk pregnant women who delivered at term by C-section, after an induced labor or after a spontaneous labor. A z-score, was calculated, referenced to cervico-vaginal fluids concentrations in subjects who delivered by C-Section. The heatmap of FIG. 11 showed clearly higher cervico-vaginal fluids levels of biomarkers in women delivering after spontaneous labor.

1. A combination of biomarkers comprising COL1A1 and CCN5 for use as biomarkers for preterm delivery, wherein the preterm delivery is a preterm delivery before 32 weeks of gestation.

2. The combination of biomarkers according to claim 1, wherein the combination of biomarkers further comprises one or more additional biomarkers for imminent birth selected from the group comprising ALDH1A1, CCL18, CCL2, CCL13, IL1RL1, CD14, CD163, TNFRSF8 or any combination thereof.

3. The combination of biomarkers according to claim 1, wherein the combination of biomarkers comprises COL1A1, CCN5, ALDH1A1, CCL18, CCL2, CCL13, IL1RL1, CD14, CD163 and TNFRSF8.

4. A method for diagnosing, whether an individual is at risk of preterm delivery and/or imminent birth by determining the presence and/or amount of a combination of biomarkers, wherein the combination of biomarkers comprises COL1A1 and CCN5.

5. The method according to claim 4, wherein the combination of biomarkers further comprises one or more additional biomarkers for imminent birth selected from the group comprising ALDH1A1, CCL18, CCL2, CCL13, IL1RL1, CD14, CD163, TNFRSF8 or any combination thereof.

6. The method according to claim 4, wherein the combination of biomarkers comprises COL1A1, CCN5, ALDH1A1, CCL18, CCL2, CCL13, IL1RL1, CD14, CD163 and TNFRSF8.

7. A method for prognosing and/or for diagnosing, whether an individual is at risk of preterm delivery and/or imminent birth, the method comprising:
a) determining the presence and/or amount of a combination of biomarkers for preterm delivery in a sample obtained from an individual, and
b) comparing the presence and/or amount of said combination of biomarkers obtained from step b) to the presence and/or amount of said combination of biomarkers in a control sample, to identify an increased risk for preterm delivery, wherein said combination of biomarkers comprises COL1A1 and CCN5.

8. The method according to claim 7, wherein the sample is a sample of vaginal fluid.

9. The method according to claim 7, wherein the combination of biomarkers for prognosing and/or for diagnosing, whether an individual is at risk of preterm delivery and/or imminent birth, further comprises one or more additional biomarkers for imminent birth selected from the group comprising ALDH1A1, CCL18, CCL2, CCL13, IL1RL1, CD14, CD163, TNFRSF8 or any combination thereof.

10. The method according to claim 7, wherein the biomarkers are protein or nucleic acid encoding for said protein.

11. The method according to claim 7, wherein the presence and/or amount of the COL1A1 and CCN5 biomarkers is determined using an antibody and/or an oligonucleotide specific for said COL1A1 and CCN5 biomarkers, and wherein the presence and/or amount of the one or more additional biomarkers is determined using an antibody and/or an oligonucleotide specific for said one or more additional biomarkers.

12. The method for prognosing and/or for diagnosing according to claim 7, whether an individual is at risk of preterm delivery and/or imminent birth, the method comprising:
a) determining the presence and/or amount of the combination of biomarkers using an antibody and/or an oligonucleotide specific for the combination of biomarkers in the sample of vaginal fluid from an individual, and
b) comparing the presence and/or amount of said combination of biomarkers obtained from step b) to the presence and/or amount of the said combination of biomarkers in a control sample, to identify an increased risk for preterm delivery and/or imminent birth,
wherein the combination of biomarkers is COL1A1, CCN5, ALDH1A1, CCL18, CCL2, CCL13, IL1RL1, CD14, CD163 and TNFRSF8.

13. The combination of biomarkers according to claim 1, wherein the individual is pregnant individual.

14. The method according to claim 4, wherein the preterm delivery is a preterm delivery before 32 weeks of gestation.

15. A device for carrying out the method according to claim 4, which comprises:
a) one or more antibody and/or oligonucleotide specific for the combination of COL1A1 and CCN5 biomarkers for preterm delivery, and optionally
b) one or more antibody and/or oligonucleotide specific for the combination of one or more additional biomarkers for imminent birth, wherein the one or more additional biomarkers is selected from the group comprising ALDH1A1, CCL18, CCL2, CCL13, IL1RL1, CD14, CD163 and TNFRSF8, and optionally
c) at least one internal standard.

16. The method according to claim 4, wherein the individual is a pregnant individual.

17. The method according to claim 7, wherein the individual is a pregnant individual.

18. The method according to claim 7, wherein the preterm delivery is a preterm delivery before 32 weeks gestation.

19. A device for carrying out the method according to claim 7, which comprises:
a) one or more antibody and/or oligonucleotide specific for the combination of COL1A1 and CCN5 biomarkers for preterm delivery, and optionally
b) one or more antibody and/or oligonucleotide specific for the combination of one or more additional biomarkers for imminent birth, wherein the one or more additional biomarkers is selected from the group comprising ALDH1A1, CCL18, CCL2, CCL13, IL1RL1, CD14, CD163 and TNFRSF8, and optionally
c) at least one internal standard.
